(19) United States
(12) Patent Application Publication
(10) Pub. No.: US 2022/0354855 A1
(43) Pub. Date: Nov. 10, 2022

(54) TREATMENT FOR GASTROINTESTINAL DISORDERS

(71) Applicant: Mayo Foundation for Medical Education and Research, Rochester, MN (US)

(72) Inventors: Purna C. Kashyap, Rochester, MN (US); Ruben Albertus Teunis Mars, Rochester, MN (US)

(21) Appl. No.: 17/762,895

(22) PCT Filed: Oct. 9, 2020
(86) PCT No.: PCT/US20/55149

§ 371 Date: Mar. 23, 2022

Related U.S. Application Data
(60) Provisional application No. 62/913,053, filed on Oct. 9, 2019.

(51) Int. Cl. A61K 31/522; A61K 35/742; A61K 35/745; A61K 35/747; A61P 1/00

(52) U.S. Cl. CPC A61K 31/522; A61K 35/742; A61K 35/745; A61K 35/747; A61P 1/00

(57) ABSTRACT

Materials and methods for treating irritable bowel syndrome (IBS) are provided herein. For example, materials and methods for increasing the level of hypoxanthine in a mammal identified as having IBS are provided herein

### CROSS-REFERENCE TO RELATED APPLICATIONS

This application claims benefit of priority from U.S. Provisional Application Ser. No. 62/913,053, filed on Oct. 9, 2019. The disclosure of the prior application is considered part of (and is incorporated by reference in) the disclosure of this application.

### STATEMENT AS TO FEDERALLY SPONSORED RESEARCH

This invention was made with government support under DK114007 awarded by the National Institutes of Health. The government has certain rights in the invention.

### TECHNICAL FIELD

This document relates to materials and methods for treating gastrointestinal disorders (e.g., irritable bowel syndrome).

### BACKGROUND

Irritable bowel syndrome (IBS) is disorder that affects 14-20% of the population in North America and is predominantly seen in females. IBS is characterized by recurrent abdominal pain or discomfort associated with changes in stool form or frequency, and is classified as constipation predominant (IBS-C), diarrhea predominant (IBS-D), or mixed based on predominant stool form.

Human studies on IBS pathogenesis implicate changes in gastrointestinal motility, secretion, visceral hypersensitivity and intestinal permeability, all of which can be modified by the gut microbiome (Bhattarai et al., Am J Physiol Gastrointest Liver Physiol 2017, 312(1):G52-G62). In addition, IBS symptoms are affected by diet, host genetics, and environment, which are also known to modulate the human gut microbiome. Experimental evidence supporting a role for the gut microbiome in IBS is based on transplantation experiments, where transit time changes associated with IBS-C and IBS-D were replicated in gnotobiotic mice following fecal microbiota transplantation from patients (Touw et al., Physiol Rep 2017, 5(6). pii: e13182. doi: 10.14814/phy2.13182; and Chiaro et al., Sci Transl Med 2017, 9(379). pii: eaaf6397. doi: 10.1126/scitranslmed.aaf6397). Similarly, a lower threshold to pain sensation characteristic of IBS patients was also seen in a rodent model following microbiota transplantation from IBS patients (Crouzet et al., Neurogastroenterol Motil 2013, 25(4): e272-282). However, in the absence of robust animal models mimicking the pathophysiology of IBS, human studies are needed to uncover the interaction of gut microbiome with relevant human-specific disease pathways of IBS.

Human studies in IBS typically are limited by small sample sizes, cross-sectional sampling, and lack of subtype stratification, all of which are reflected in the lack of agreement in findings across the large number of microbiome studies in IBS patients (Duan et al., Clin Transl Gastroenterol 2019, 10(2):1-12). Like other chronic gastrointestinal disorders, IBS is characterized by periods of remission and exacerbation in symptoms, and thus a cross-sectional sample fails to account for the temporal variability. The lack of subtype stratification further increases variability given the well-described influence of gastrointestinal transit on the gut microbiome (Nature Microbiol 2016, 1: 16093, and Kashyap et al., Gastroenterol 2013, 144(5):967-977). Finally, the disconnect between human and animal studies has been a major barrier in advancing understanding of the role of the gut microbiome in IBS.

### SUMMARY

As described herein, multi-omics integration studies were conducted to determine pathways relevant in pathogenesis of IBS. This document is based, at least in part, on the discovery that a consistent decrease in hypoxanthine is observed in the stool of patients with IBS, along with increased expression of xanthine oxidase in the intestinal epithelial cells and the gut bacteria. This document also is based, at least in part, on the identification of up-regulated genes involved in the purine salvage pathway in samples from IBS patients, reflecting purine starvation in the epithelial tissue. Thus, this document provides materials and methods for improving gastrointestinal epithelial function and/or treating gastrointestinal disorders (e.g., functional gastrointestinal disorders such as IBS). In some cases, the materials and methods described herein can be used to reduce symptoms of IBS by increasing hypoxanthine levels (e.g., by administering hypoxanthine itself or by reducing the degree of hypoxanthine metabolism, such as by inhibiting xanthine oxidase).

In a first aspect, this document features a method for treating a mammal identified as having IBS. The method can include treating the mammal to increase hypoxanthine levels in the mammal. The treating can include comprises administering to the mammal an agent effective to increase levels of hypoxanthine in the mammal. The agent can be hypoxanthine. The agent can be an inhibitor of xanthine oxidase (e.g., allopurinol). The agent can include at least one live bacterial organism having the ability to produce hypoxanthine (e.g., Escherichia coli K12, an Enterococcus sp, a Faecalibacterium sp, a Bacillus sp., or Bacteroides thetaiotaomicron engineered to produce hypoxanthine). The treating can include removing from the mammal at least one bacterial organism having xanthine oxidase activity (e.g., a Lachnospiraceae spp. or Hungatella hathewayi). The method can include selectively removing the at least one bacterial organism by administering a bacteriophage (e.g., a naturally occurring or engineered bacteriophage) or an antimicrobial compound (e.g., a lantibiotic).

The method can further include administering to the mammal at least one live bacterial organism having tryptophan decarboxylase activity (e.g., a Prevotella sp., a Bacteroides sp., a Clostridium sp., a Faecalibacterium sp., a Eubacterium sp., a Ruminococcus sp., a Peptococcus sp., a Peptostreptococcus sp., a Bifidobacterium sp., an Escherichia sp., a Lactobacillus sp., an Akkermansia sp., or a Roseburia sp.). In some cases, the at least one live bacterial organism can be Ruminococcus gnavus or Clostridium sporogenes.

The method can further include administering to the mammal at least one live bacterial organism having the ability to produce short chain fatty acids (e.g., acetate and/or butyrate). The at least one live bacterial organism can be Faecalibacterium prausnitzii (clostridial cluster IV), an Anaerostipes sp., a Eubacterium sp., a Roseburia sp. (clostridial cluster XIVa), a Blautia sp., a Bifidobacteria sp., a Lactobacillus sp., Akkermansia muciniphila, a Prevotella sp., or a Ruminococcus sp.

The method can further include administering to the mammal at least one live bacterial organism having the ability to convert primary bile acids to secondary bile acids. The at least one bacterial organism can have the ability to convert cholic acid to deoxycholic acid and/or the ability to convert chenodeoxycholic acid to lithocholic acid. The at least one bacterial organism can be a Clostridium sp. (e.g., Clostridium scindens or an engineered Clostridia sp.).

The mammal can be a human, non-human primate, cow, pig, horse, dog, cat, rat, or mouse. The agent can be administered orally or rectally. The agent can be formulated in a capsule, liquid, suppository, enema, or food product. The administering can be effective to reduce one or more symptoms of IBS in the mammal.

In another aspect, this document features a method for identifying a mammal as having IBS. The method can include measuring the level of hypoxanthine in a biological sample obtained from the mammal, and determining that the measured level of hypoxanthine is less than a control level of hypoxanthine in one or more corresponding mammals that do not have IBS. The measuring can include using ¹H-NMR, LC-MS, or a xanthine/xanthine oxidase assay.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

DESCRIPTION OF DRAWINGS

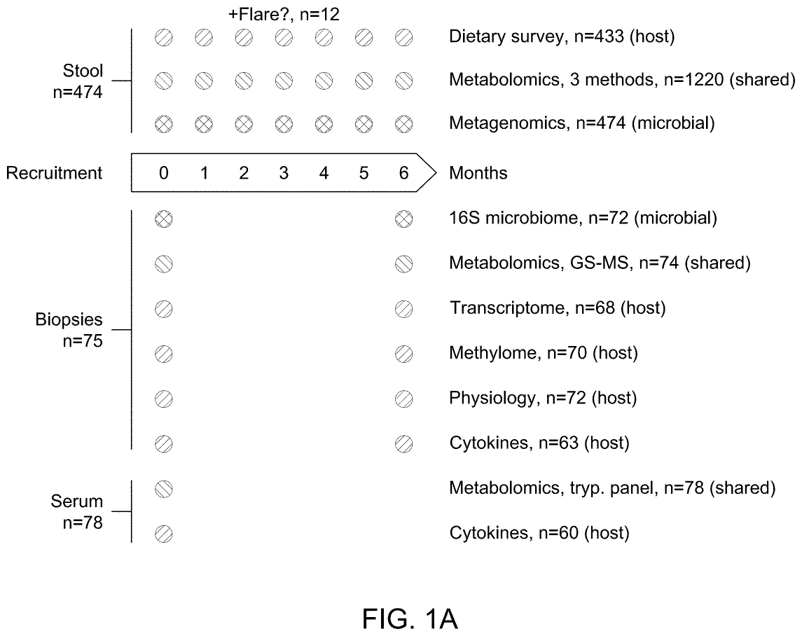

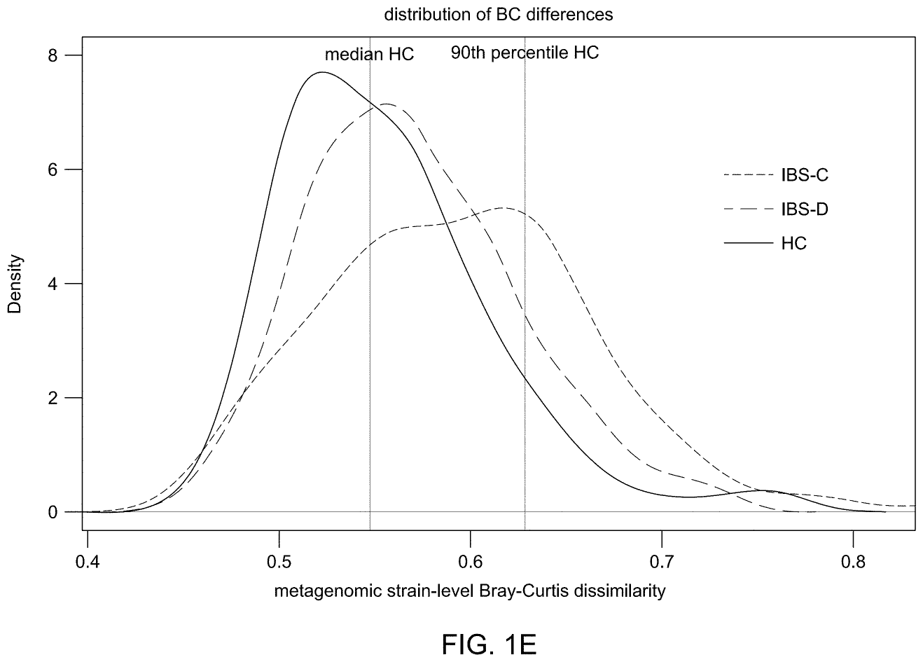

FIGS. 1A-1I: Gut microbiota composition is variable in IBS-C patients. FIG. 1A is an outline of the study and characteristics of recruited subjects described in the Examples herein. FIG. 1B is a diagram showing the timeline of paired longitudinal samples per subject. In particular, FIG. 1B is a Beta diversity ordination (Bray Curtis) showing distribution of samples from IBS-C (C), IBS-D (D), and healthy controls (H), either considering all samples from all subjects (FIG. 1C) or considering by-subject averaged data (FIG. 1D). PERMANOVA on group membership. FIG. 1E is a graph plotting a Bray Curtis Dissimilarity Index (BCDI) showing the distribution of the three groups (mixed linear models correcting for subject p-value HC vs. IBS-C<0.01). FIG. IF is a Beta diversity ordination (Bray Curtis) showing distribution of biopsy and stool samples from the subgroups (PERMANOVA on group membership). FIG. 1G is a graph plotting community variability within each group based on mean Bray Curtis Distance. FIG. 1H is a graph plotting the difference in mucosa associated and luminal microbiota composition based on distance metric (ANOVA Tukey HSD p-values<0.001). FIG. 1I is a graph plotting community variability within each group based on mean Bray Curtis Distance.

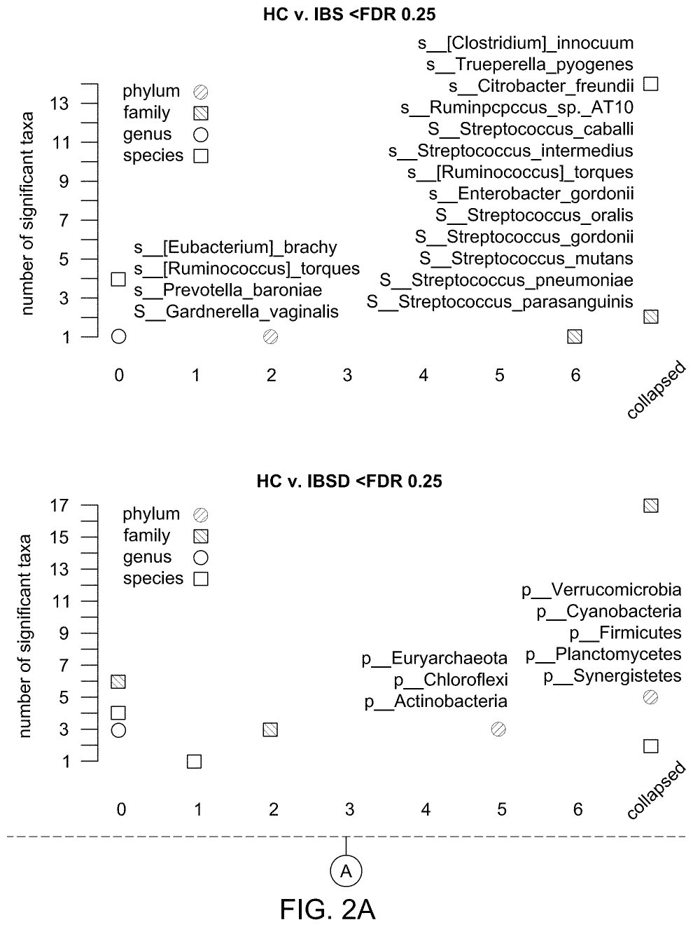

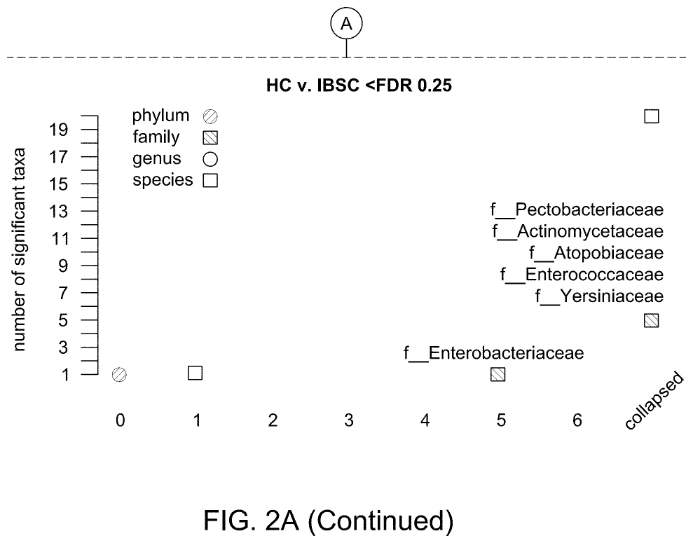

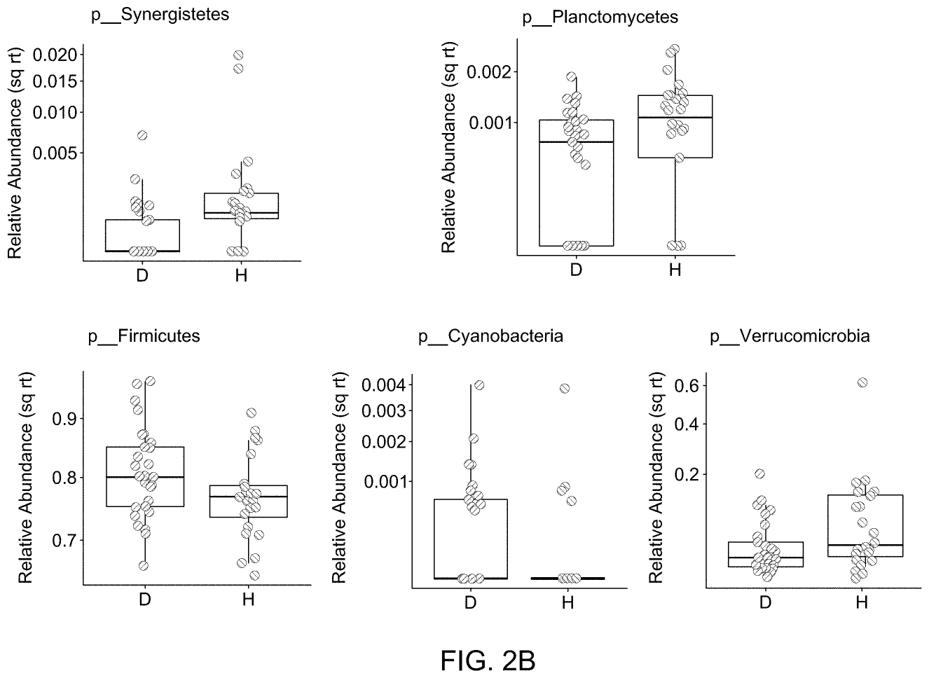

FIGS. 2A-2C: Longitudinal sampling overcomes heterogeneity seen across cross-sectional microbiome studies. FIG. 2A shows significantly different taxa that were identified at FDR<0.25 (Mann-Whitney test) when comparing HC with all samples from IBS (top), comparing HC with IBS-C (middle), or comparing HC with IBS-D (bottom) at three individual time points (0, 2, and 6 months) and collapsed data including all time points. Symbols indicate different taxonomic levels. FIG. 2B is a series of representative plots showing the abundance of the indicated five phyla that are significantly different when comparing IBS-C and HC. FIG. 2C is a pair of representative plots showing the abundance of Proteobacteria in colonic biopsies obtained from IBS-C and HC at two different time points (T1, n=29, 13 respectively; T2, n=21, 9 respectively).

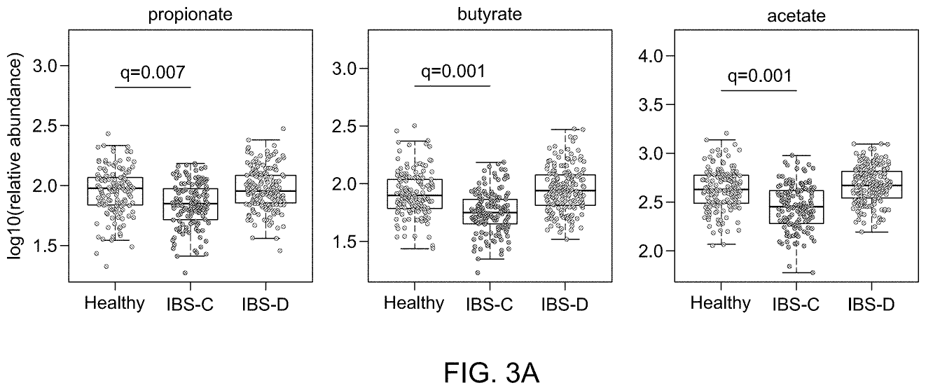

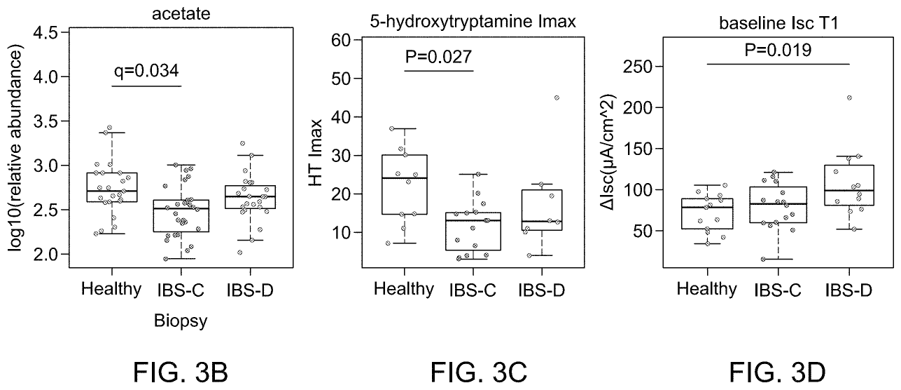

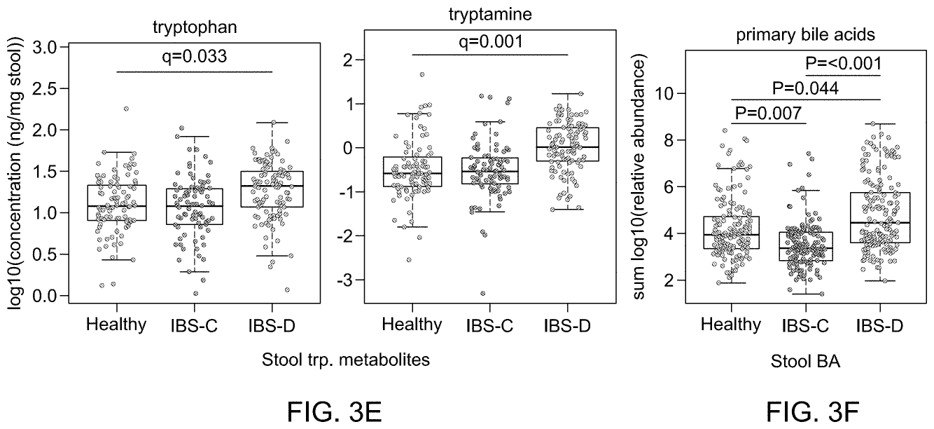

FIGS. 3A-3F: Integrated top-down bottom-up approach provides mechanistic insight into the effect of gut microbiota metabolism on host physiology. FIG. 3A is a series of graphs plotting the abundance of propionate (left), butyrate (center), and acetate (right) as determined by ¹H NMR. The data are shown as mixed linear models correcting for subject in stool samples from healthy, IBS-C, and IBS-D subjects. FIG. 3B is a graph plotting the abundance of acetate in colonic biopsies. FIG. 3C is a graph plotting maximal ΔIsc (Imax) following application of cumulative concentrations of serotonin (5-HT) basolaterally in colonic biopsies from healthy, IBS-C, and IBS-D subjects. FIG. 3D is a graph plotting baseline Isc observed in colonic biopsies from healthy, IBS-C, and IBS-D subjects (P-values are Tukey HSD adjusted from a linear model). FIG. 3E is a pair of graphs plotting the abundance of tryptophan (left) and tryptamine (right) in stool samples from healthy, IBS-C, and IBS-D subjects. FIG. 3F is a graph plotting the abundance of primary unconjugated bile acids in stool samples from healthy, IBS-C and IBS-D subjects.

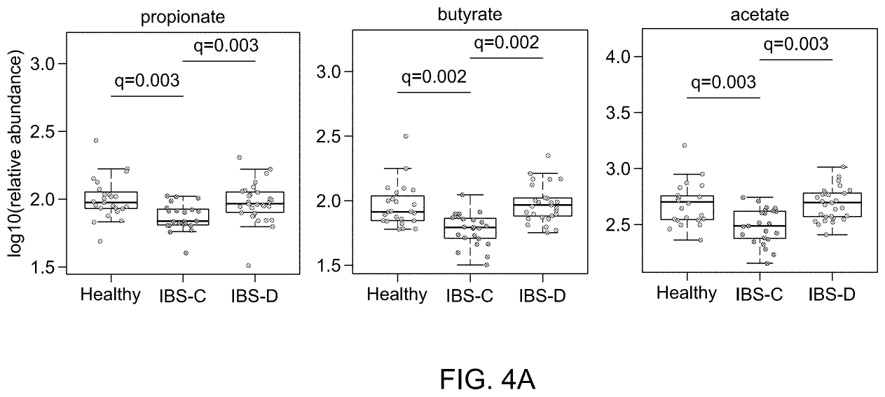

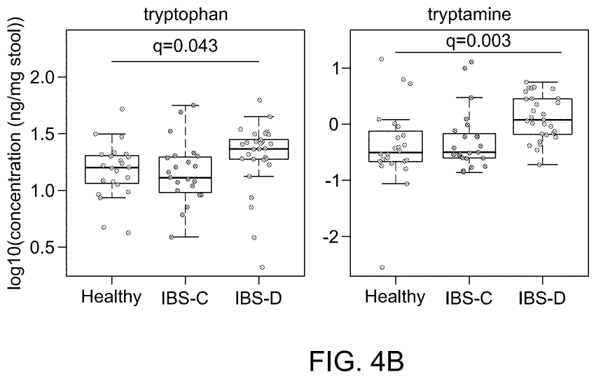

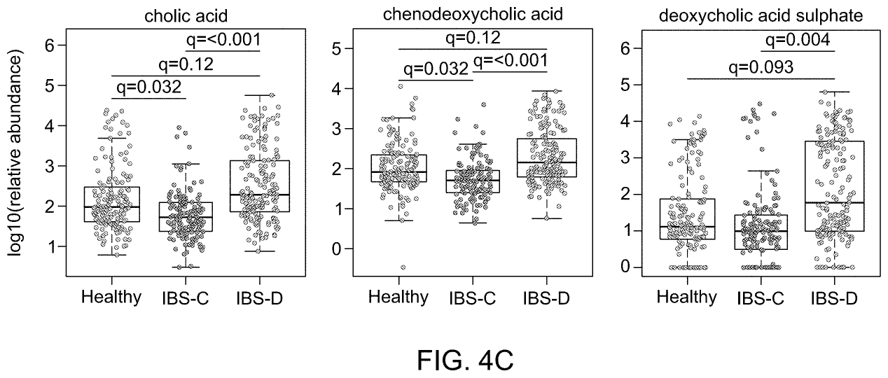

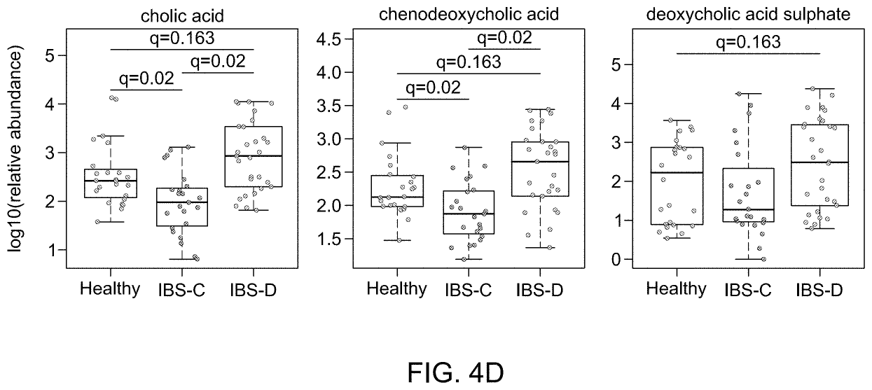

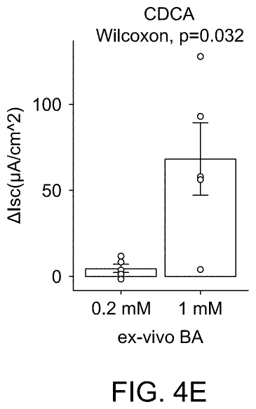

FIGS. 4A-4E: An integrated metabolomics approach provides mechanistic insight into the effect of gut microbiota metabolism on host physiology. FIG. 4A is a series of graphs plotting the abundance of propionate (left), butyrate (center), and acetate (right) as determined by ¹H NMR using stool samples from healthy, IBS-C, and IBS-D subjects (averaged data per subject, pairwise Mann-Whitney tests). FIG. 4B is a pair of graphs plotting the abundance of tryptophan (left) and tryptamine (right) as determined by LC-MS in stool samples from healthy, IBS-C, and IBS-D subjects (averaged data per subject, pairwise Mann-Whitney tests). FIG. 4C is a series of graphs plotting the abundance of cholic acid (CA, left panel), chenodeoxycholic acid (CDCA, center panel), and deoxycholic acid sulfate (DCA-S, right panel) as determined by ¹H NMR with stool samples from healthy, IBS-C, and IBS-D subjects (mixed linear models correcting for subject). FIG. 4D is a series of graphs plotting the abundance of CA, CDCA, and DCA-S as determined by ¹H NMR using stool samples from healthy, IBS-C, and IBS-D subjects (averaged data per subject, pairwise Mann-Whitney tests). FIG. 4E is a graph plotting the ΔIsc (Imax) following application CDCA basolaterally in proximal colon mucosa submucosa preparations from germ free mice (n=3, Wilcoxon).

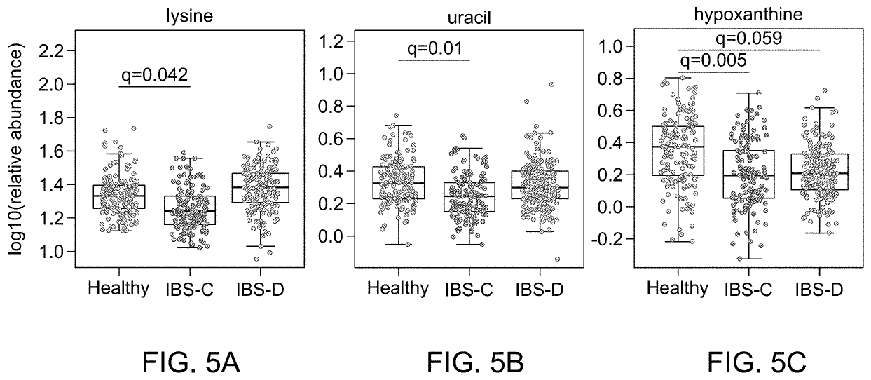

FIGS. 5A-5D: Integrated microbiome-metabolome analysis identifies a novel microbial metabolic pathway in IBS. FIGS. 5A-5C are graphs plotting the abundance of lysine (FIG. 5A), uracil (FIG. 5B), and hypoxanthine (FIG. 5C) in stool samples from healthy, IBS-C, and IBS-D subjects (determined by ¹H NMR, mixed linear models correcting for subject). FIG. 5D is a series of graphs plotting hypoxanthine-related KO terms based on metagenomic analysis of stool samples from healthy and IBS-C subjects. Data from collapsed microbiome data is plotted (all FDR<0.1, Mann-Whitney test).

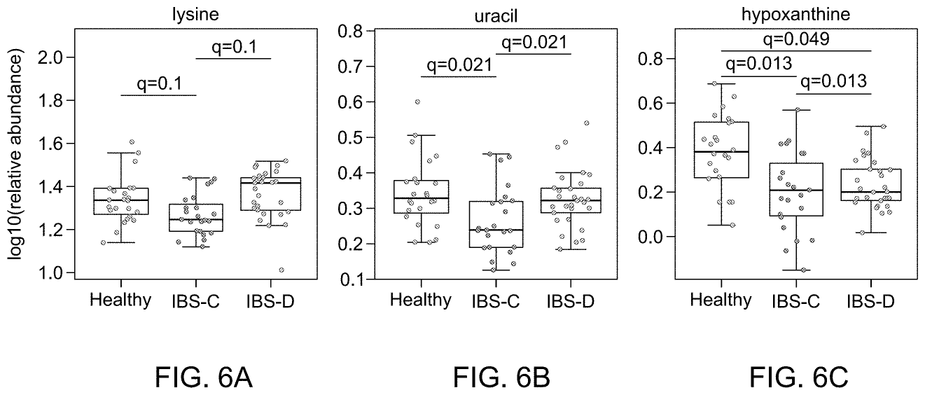

FIGS. 6A-6C: Integrated microbiome-metabolome analysis identifies a novel microbial metabolic pathway in IBS. FIGS. 6A-6C are graphs plotting the abundance of lysine (FIG. 6A), uracil (FIG. 6B), and hypoxanthine (FIG. 6C) in stool samples from healthy, IBS-C, and IBS-D subjects (determined by ¹H NMR, averaged data per subject, pairwise Mann-Whitney tests).

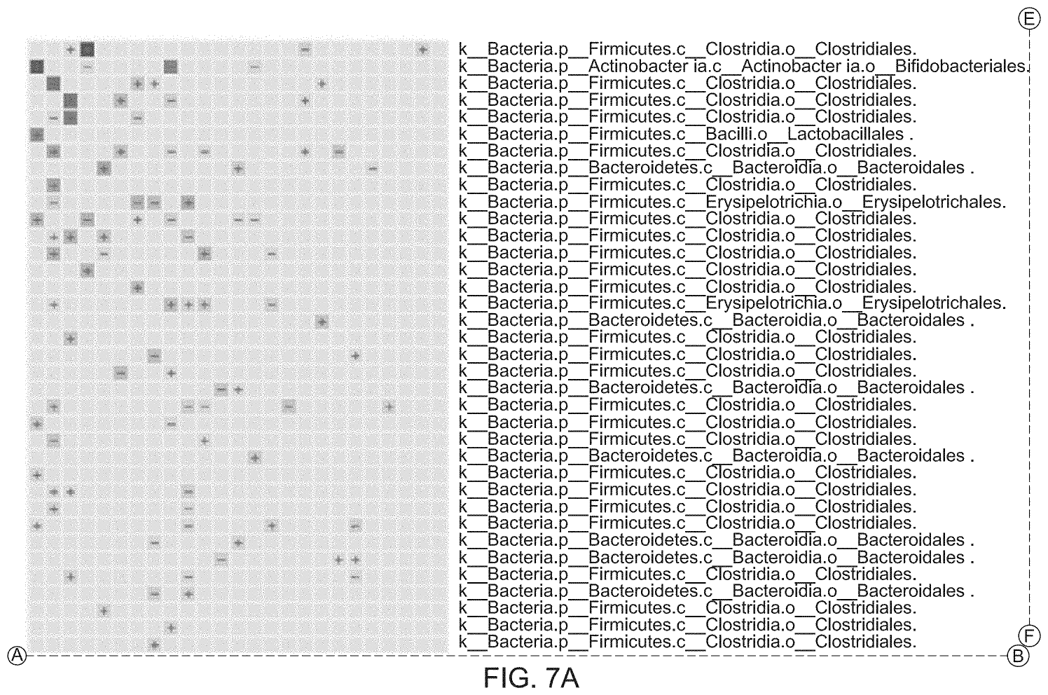

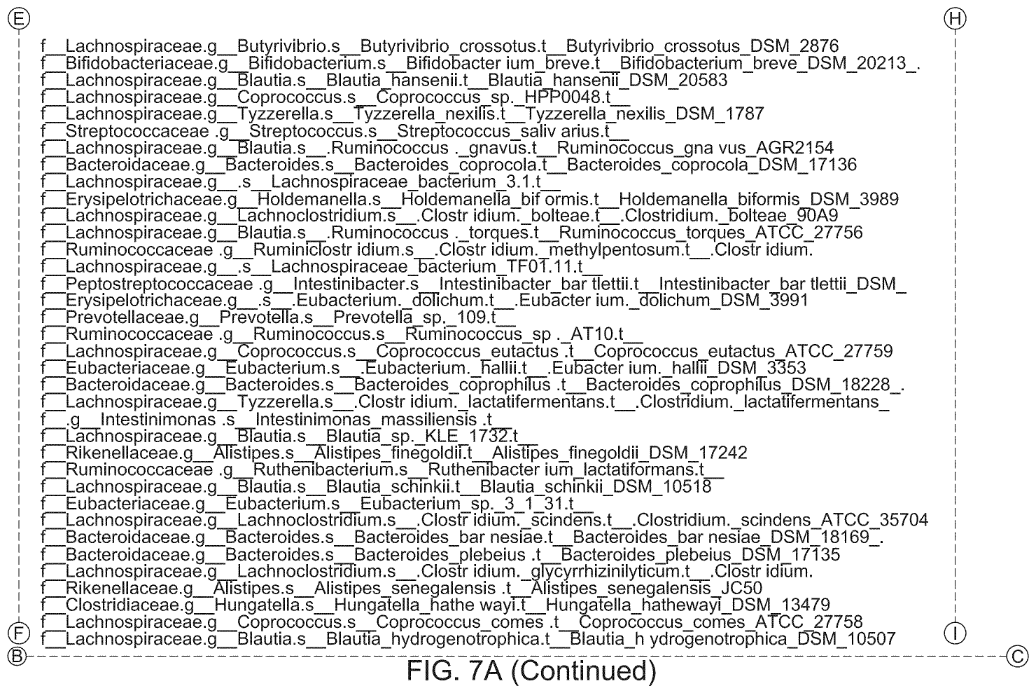

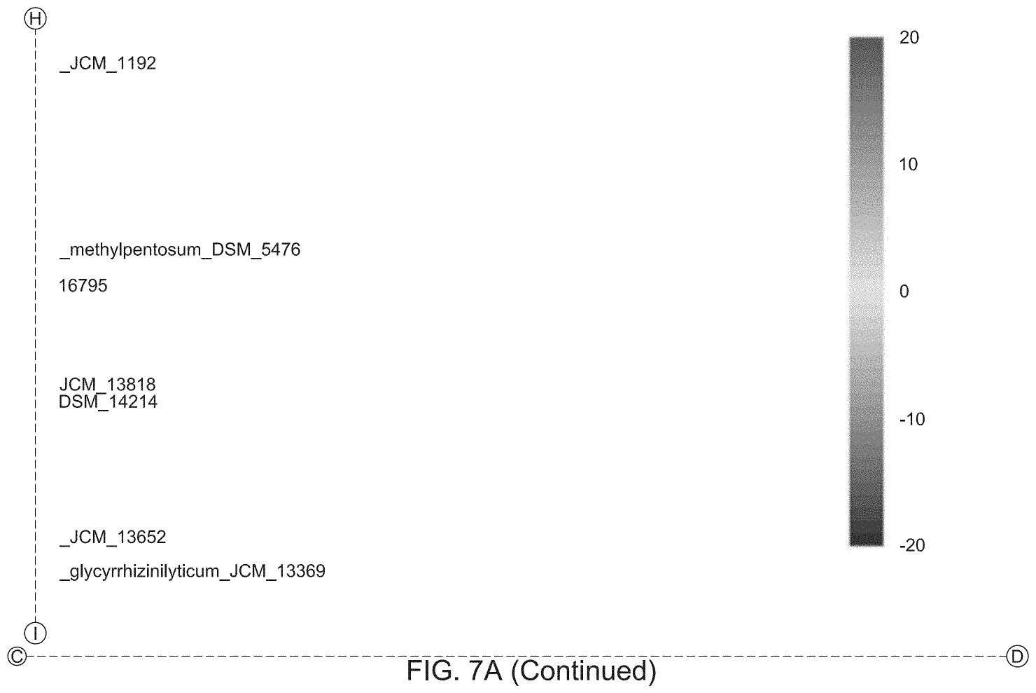

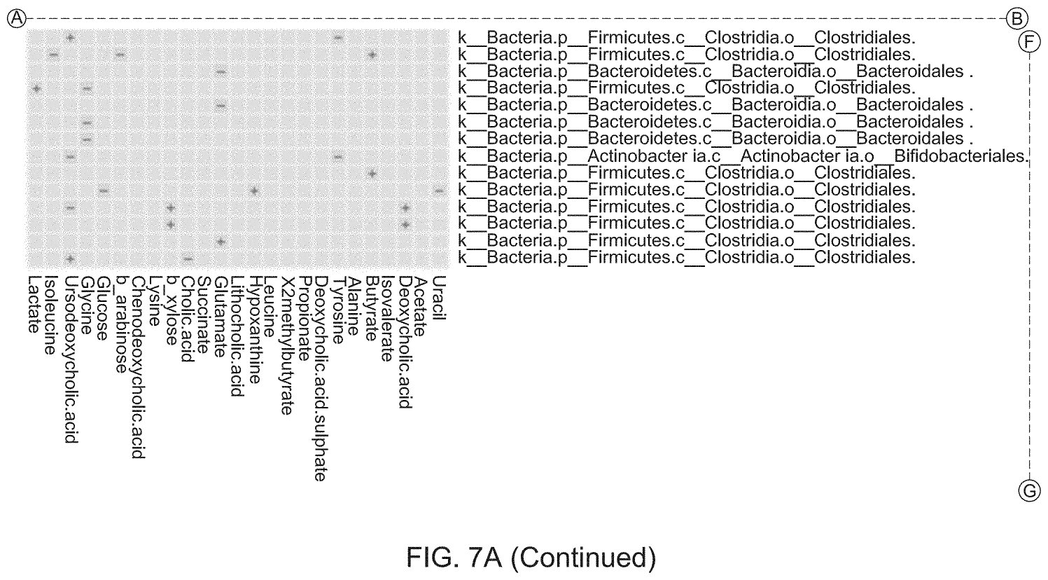

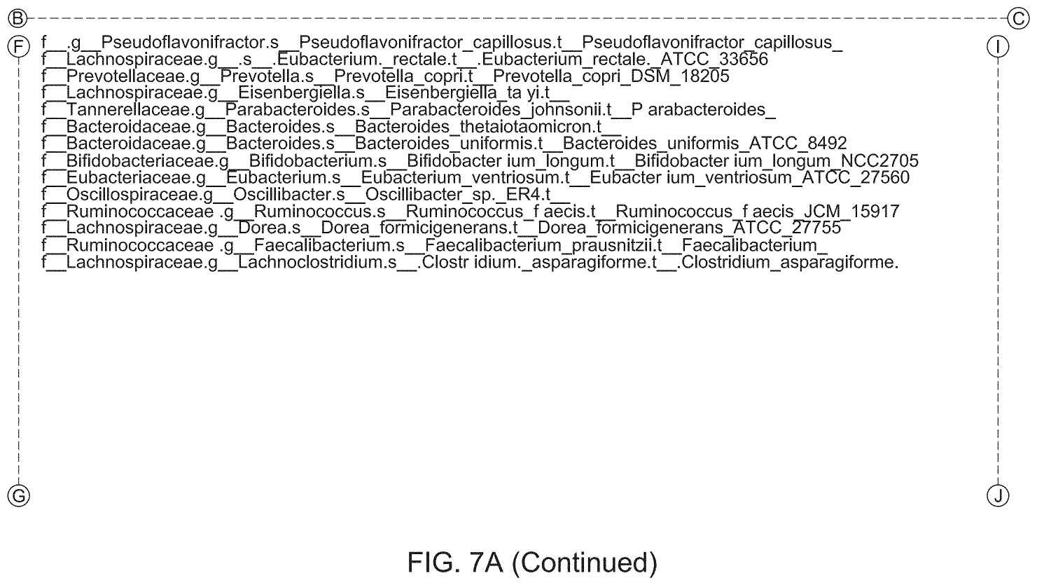

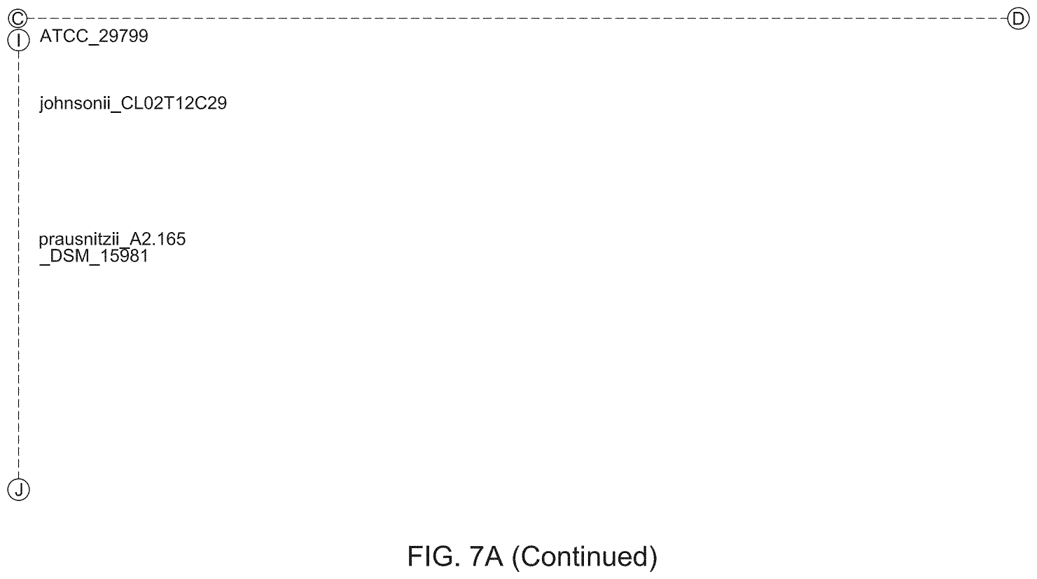

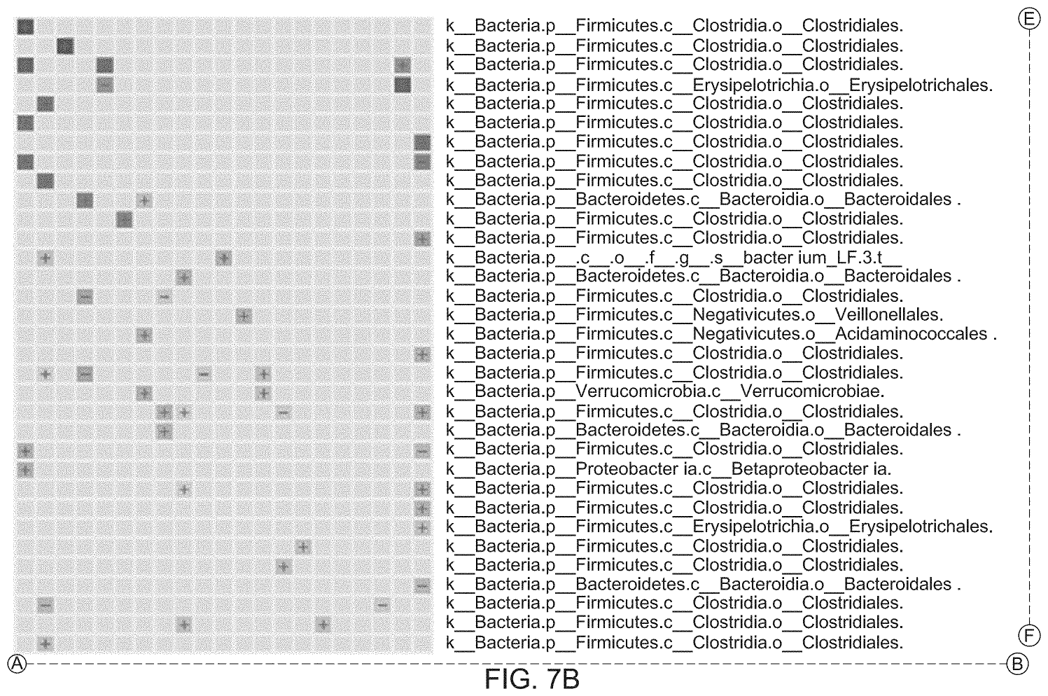

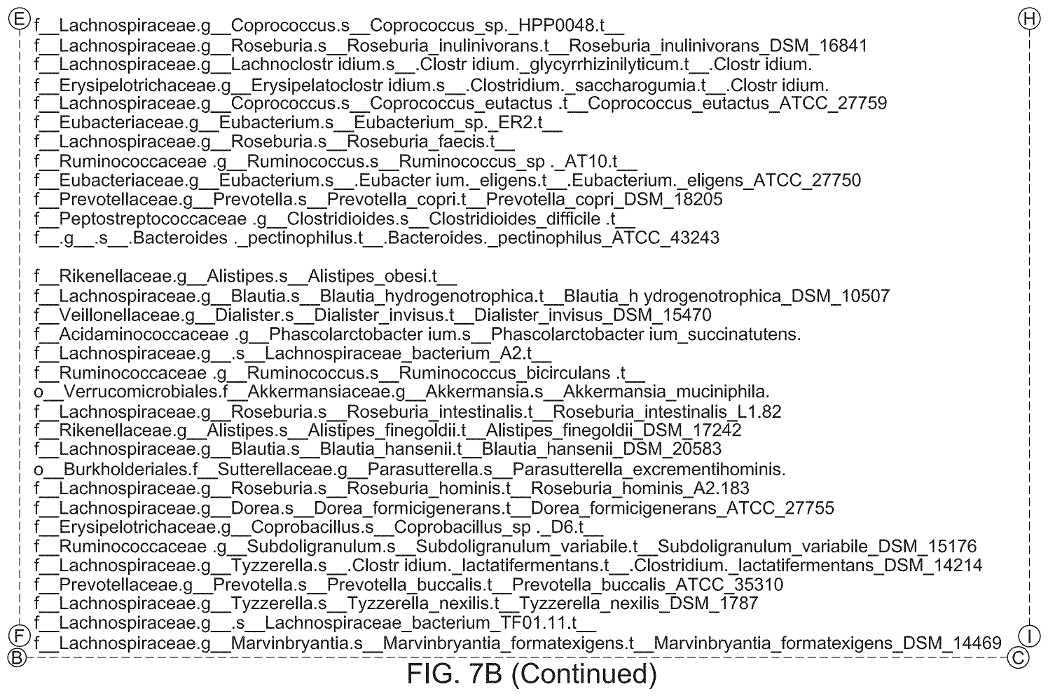

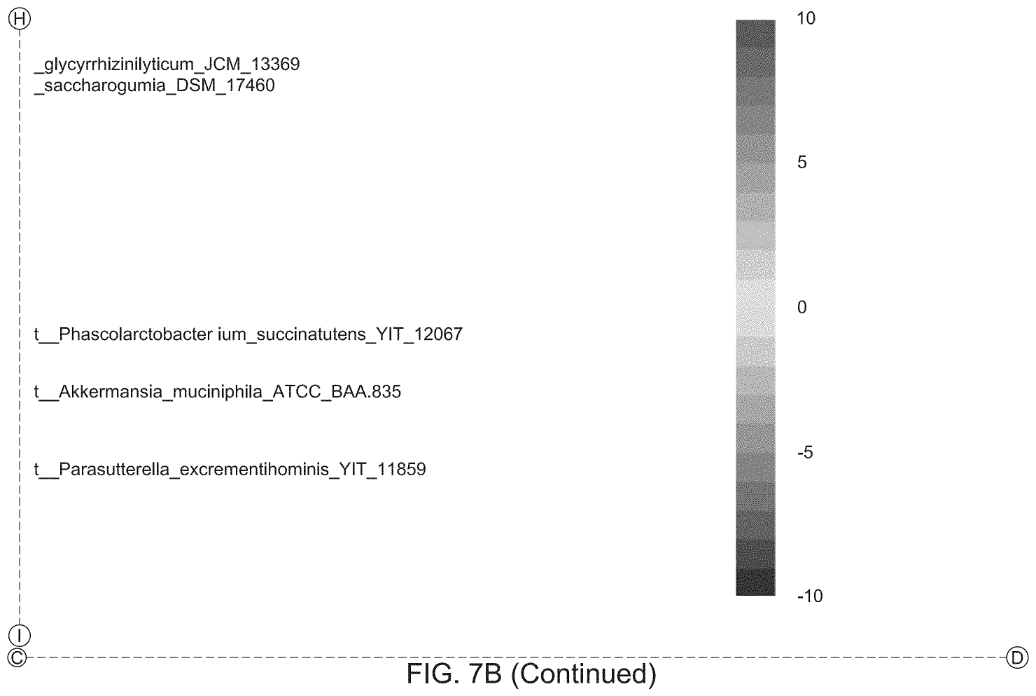

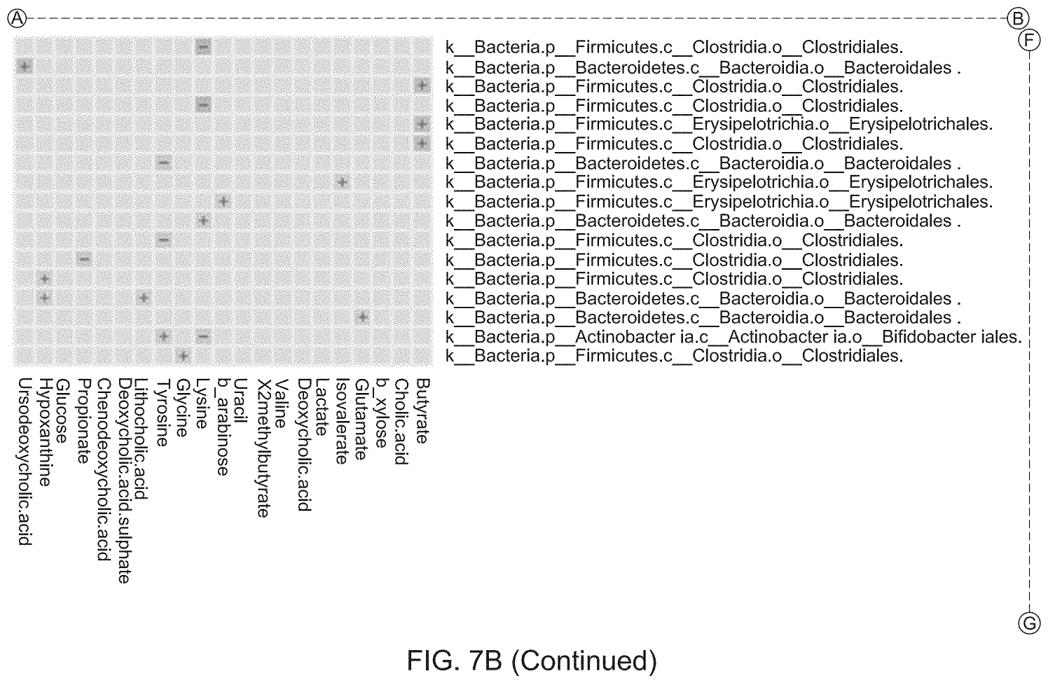

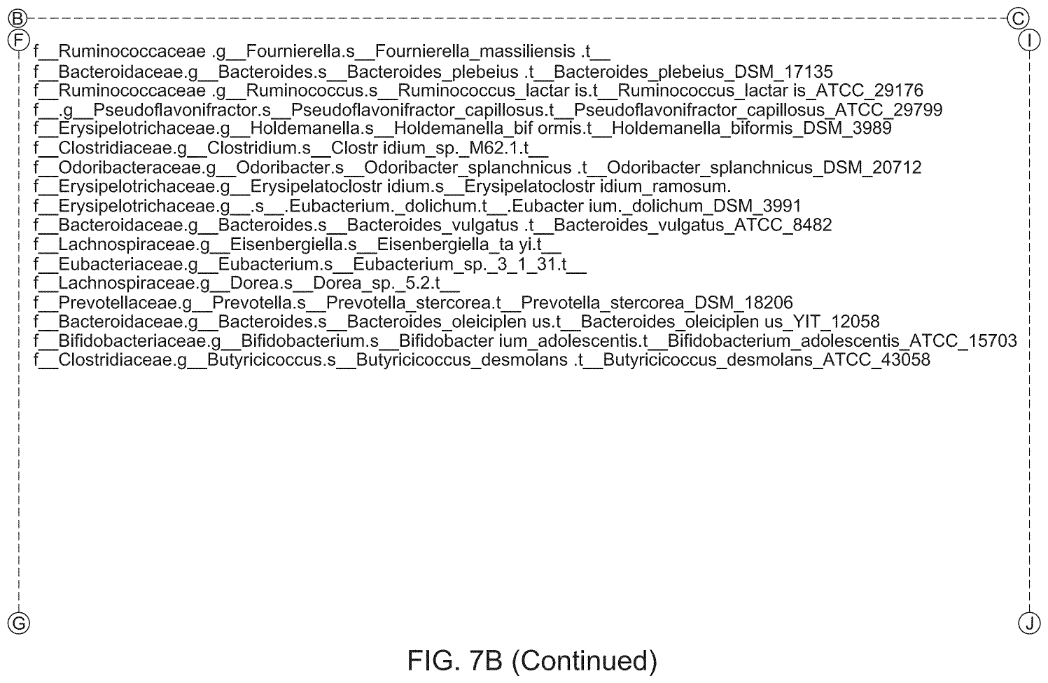

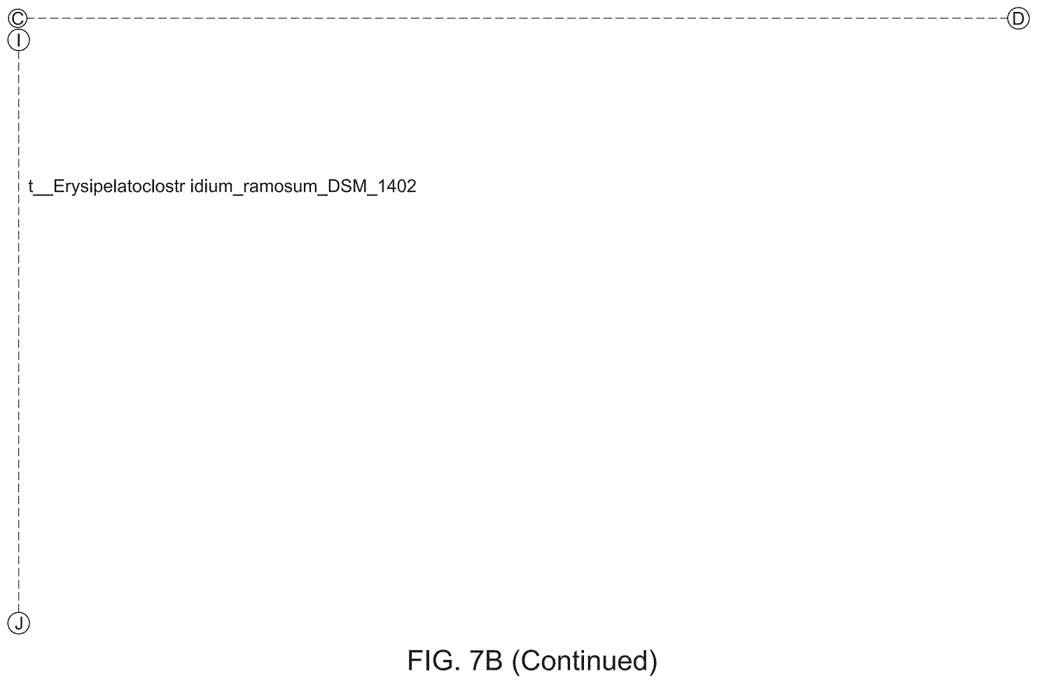

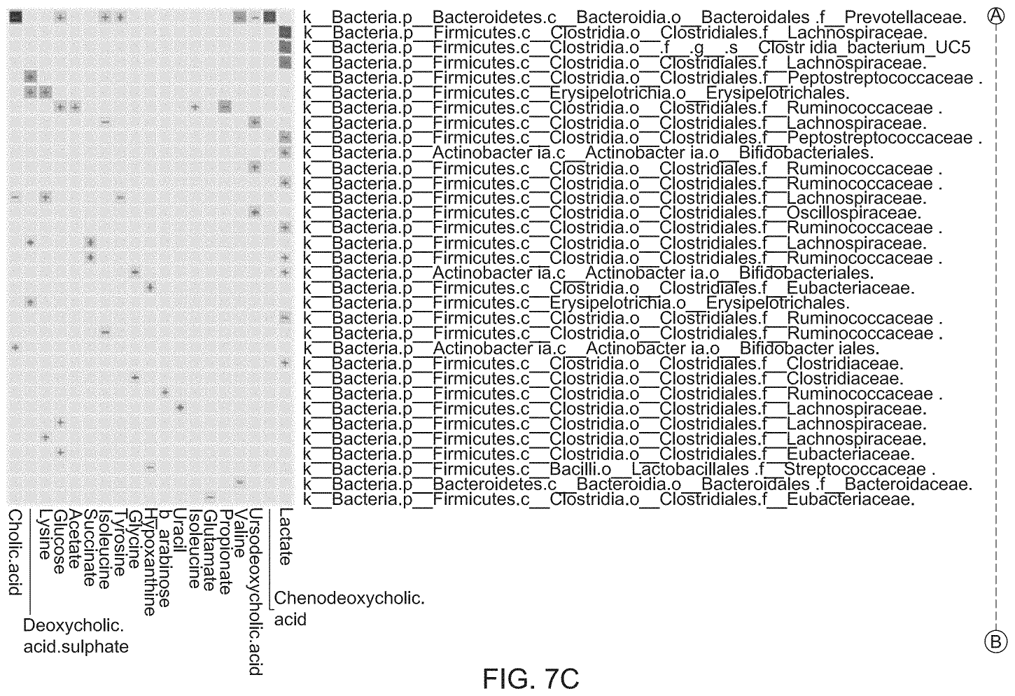

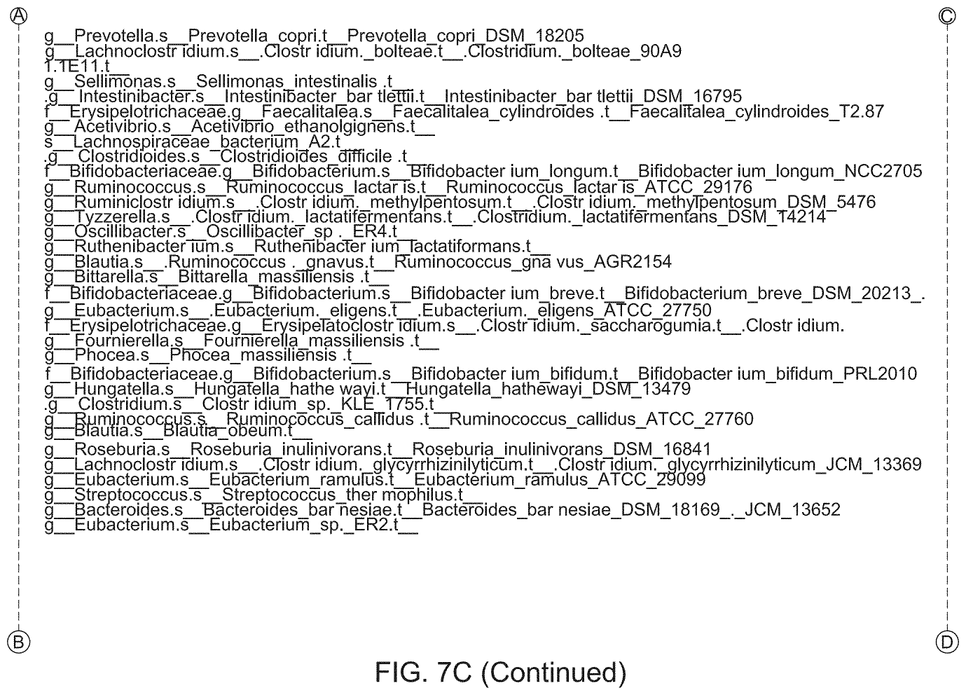

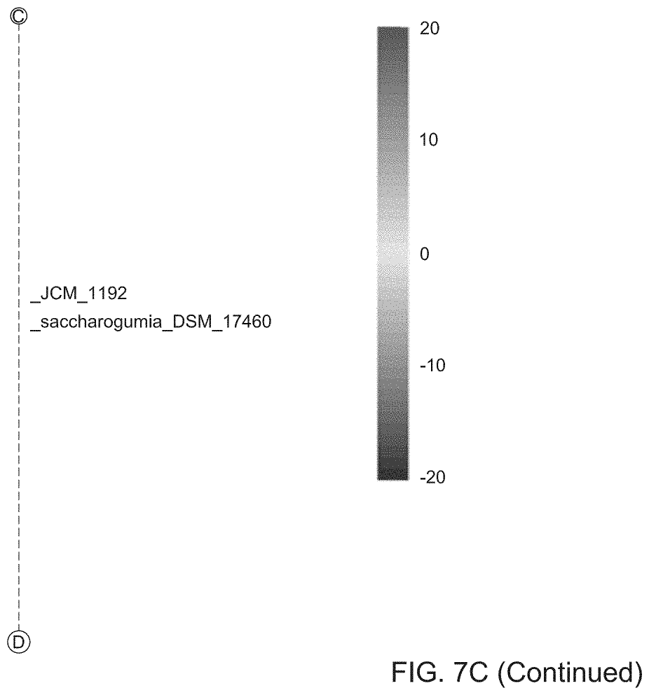

FIGS. 7A-7E: Multivariate correlation analysis based on linear models (Maaslin) to identify microbe-metabolite correlations. FIGS. 7A-7C are heatmaps showing correlation of stool microbiome with luminal metabolome in stool samples from healthy controls (FIG. 7A), IBS-D (FIG. 7B), and IBS-C (FIG. 7C). The top 50 features with −log(qval)*sign(coeff) below Maaslin cutoff are shown. FIGS. 7D and 7E are graphs plotting the correlation of Eubacterium eligens and hypoxanthine identified in stool samples from IBS-D (FIG. 7D) and IBS-C (FIG. 7E).

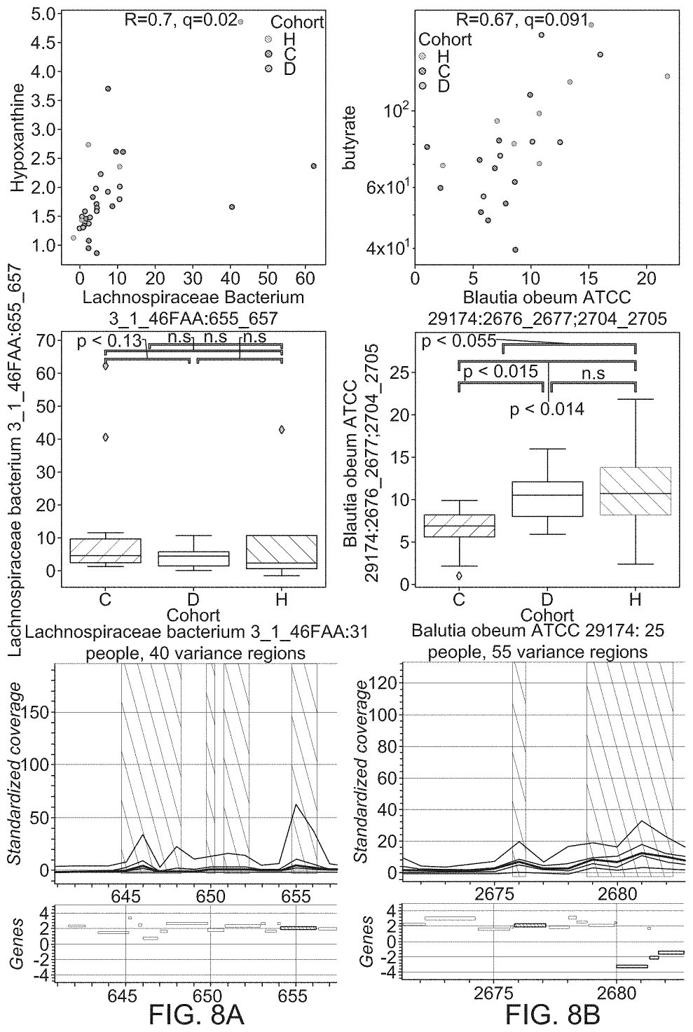

FIGS. 8A-8B: Microbial gene regions contributing to the differences in microbial metabolites in IBS. FIG. 8A is a series of plots showing that a genomic region of Lachnospiraceae bacterium 3-146FAA positively correlates to hypoxanthine. pBLAST analysis perfectly matches the coding sequence (CDS) with topoisomerase III (E value=0). The region is not significantly different between the subgroups. The different panels present, from top to bottom, a scatter plot of intensities and region abundances, statistical comparison or region abundance across cohorts, and genomic context. FIG. 8B is a series of plots showing that Blautia obeum ATCC 29174 genomic regions positively correlate to butyrate. These regions are significantly lower in IBS-C, which is in line with its lower butyrate levels. Region 2676-2677 contains a tetricoat peptide and region 2704-2705 contains a type III ribonuclease. The panels are as in FIG. 8A.

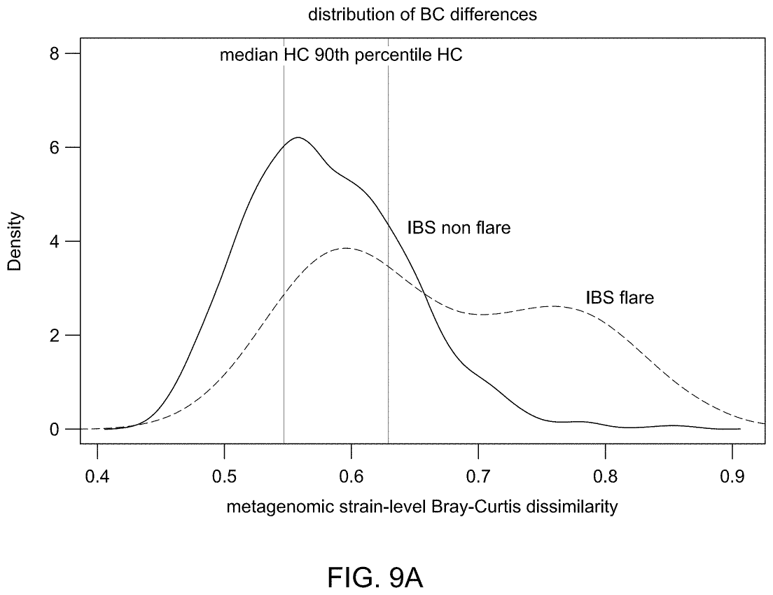

FIGS. 9A-9F: Alteration in gut microbiome and microbial metabolites underlie flares in IBS patients. FIG. 9A is a BCDI plot showing distribution of the flare and averaged non flare samples (mixed linear models correcting for subject; p-value 0.011). FIG. 9B is a graph demonstrating that BCDI score within-disease flare comparisons showed significantly higher BCDI. P-values for FIGS. 9A and 9B are from linear mixed-effect model correcting for subject. FIG. 9C is a graph showing that flares had significantly lower alpha diversity compared to the subject averages (p-values from Mann-Whitney test). FIG. 9D is a graph plotting the relative abundance of Halobiforma nitratireducens in flare and non-flare IBS samples (q-value<0.001, Mann-Whitney test). FIGS. 9E and 9F are graphs plotting the relative abundance of CA (FIG. 9E) and CDCA (FIG. 9F) in stool samples from IBS-C (flare and non-flare), IBS-D (flare and non-flare), and HC, as determined by ¹H NMR (q-values from linear mixed-effect models correcting for subject).

FIGS. 10A-10D: Alteration in gut microbiome and microbial metabolites underlie flares in IBS patients. FIG. 10A is a graph plotting the time-dependence of Bray-Curtis dissimilarity (BCD) for the microbiome of subject 10007572. The black (curved) line is a spline fit, and the grey (horizontal) lines indicate the median and 90th percentile of median HC dissimilarities. The open circle indicates flare (p-value from perturbation analysis<0.01). FIG. 10B is a graph plotting the Bray-Curtis dissimilarity (BCD) of the microbiome for subject 10007572. The flare sample stood out from the other samples. FIG. 10C is a list correlating BCD with microbiome species abundance for the subject identified bacteria strongly associated to flares. FIG. 10D is a series of graphs showing that the flare sample stands out in tryptamine (top), CA (second from top), CDCA second from bottom), and the bile salt hydrolase module (bottom). Grey lines indicate Z-scores at alpha level of 0.05 (|Z|=1.645). Orange: flare sample.

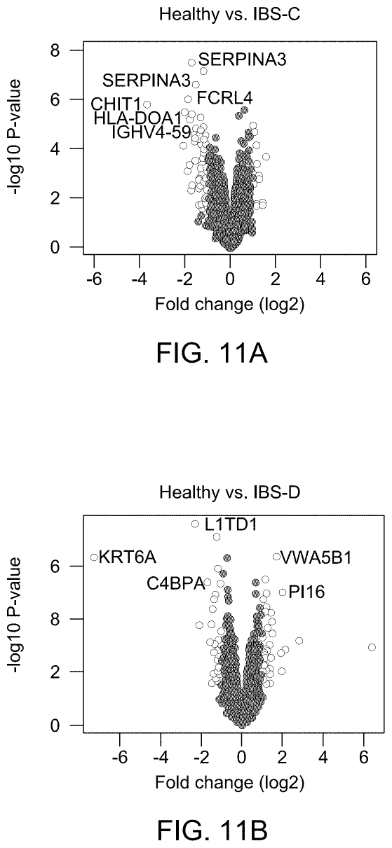

FIGS. 11A-11E: Epigenetic and transcriptomic changes in colonic biopsies as a measure of host physiologic state in IBS. FIGS. 11A and 11B are volcano plots highlighting differentially expressed (DE) genes when comparing HC and IBS-C (FIG. 11A), or HC and IBS-D (FIG. 11B). Significant DE genes are highlighted as gray. FIG. 11C is a Venn diagram displaying overlap between significantly DE genes (2 fold change, p-value<0.05) comparing HC and IBS-C (HvC) and HC and IBS-D (HvD). FIG. 11D is a Venn diagram showing overlap in differentially methylated regions (DMR) comparing HC and IBS-C (HvC) and HC and IBS-D (HvD). FIG. 11E is a table listing the results from KEGG pathway enrichment analyses of significant DE and DMR genes, comparing HC and IBS-C and HC and IBS-D.

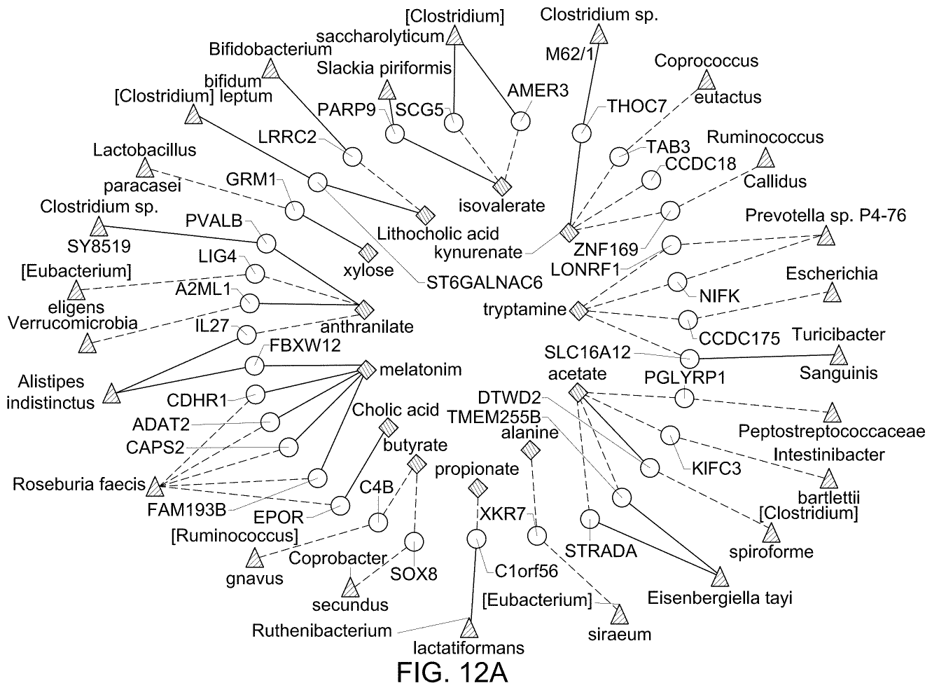

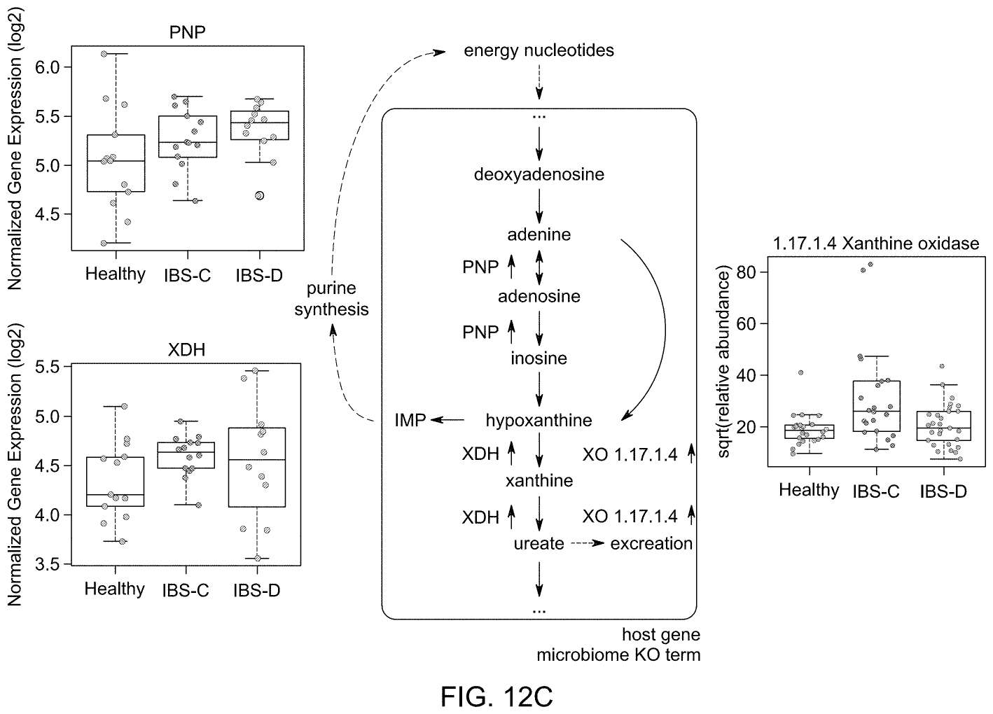

FIGS. 12A-12C: An integrated multi-omics view of IBS points to microbiome-host interactions. FIG. 12A is a schematic of a network representing significant and stability-selected correlations of host genes (round nodes) with fecal taxa (triangular nodes) and fecal metabolites (diamond nodes) at FDR<0.25 using Limma correlation. Solid lines indicate positive correlation and dashed lines indicate negative correlation, while line width indicates the strength of correlation. FIG. 12B is a series of Limma plots showing the negative correlation between acetate and expression of the PGLYPR1 (top) and KIFC3 (middle) genes, and between hypoxanthine and PNP expression (bottom). FIG. 12C includes a diagram illustrating the purine salvage pathway (shaded grey box) with associated changes observed in the presently described studies. PNP (upper left) and XDH (lower left) expression was elevated in both IBS-C and IBS-D at one or both of the biopsy time points. Data from time point 1 is plotted (all PNP p-values<0.001, XDH 0.02 for IBS-C and 0.10 for IBS-D in time point 1, and <0.005 for time point 2 comparisons). The metagenomic xanthine oxidase module abundance is shown for all groups (enter right; replotted from FIG. 5D but for all groups). IBS-C vs HC FDR<0.1, Mann-Whitney test.

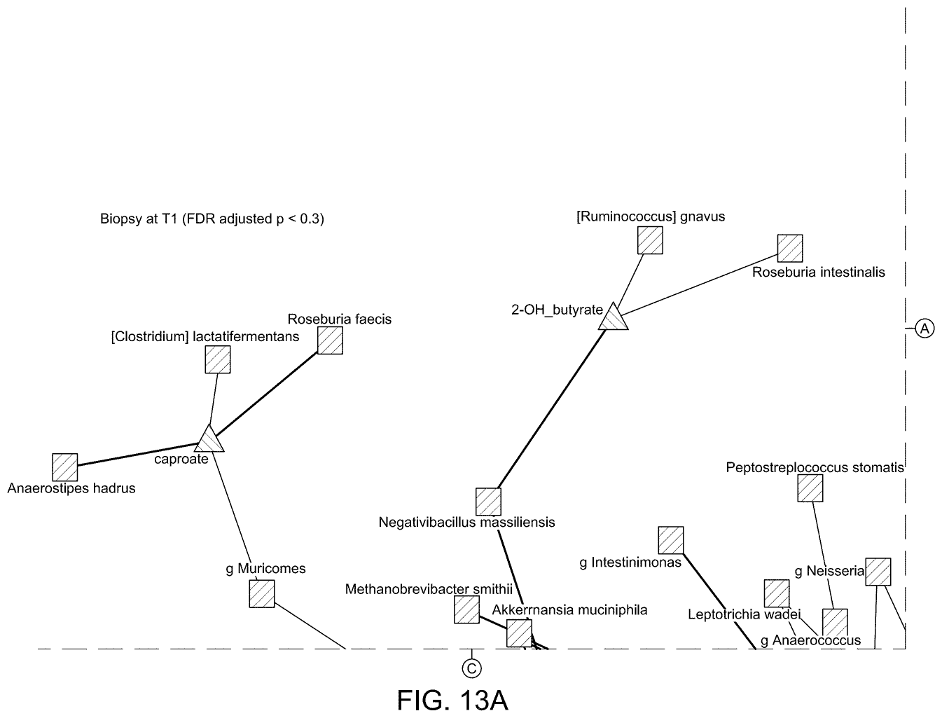

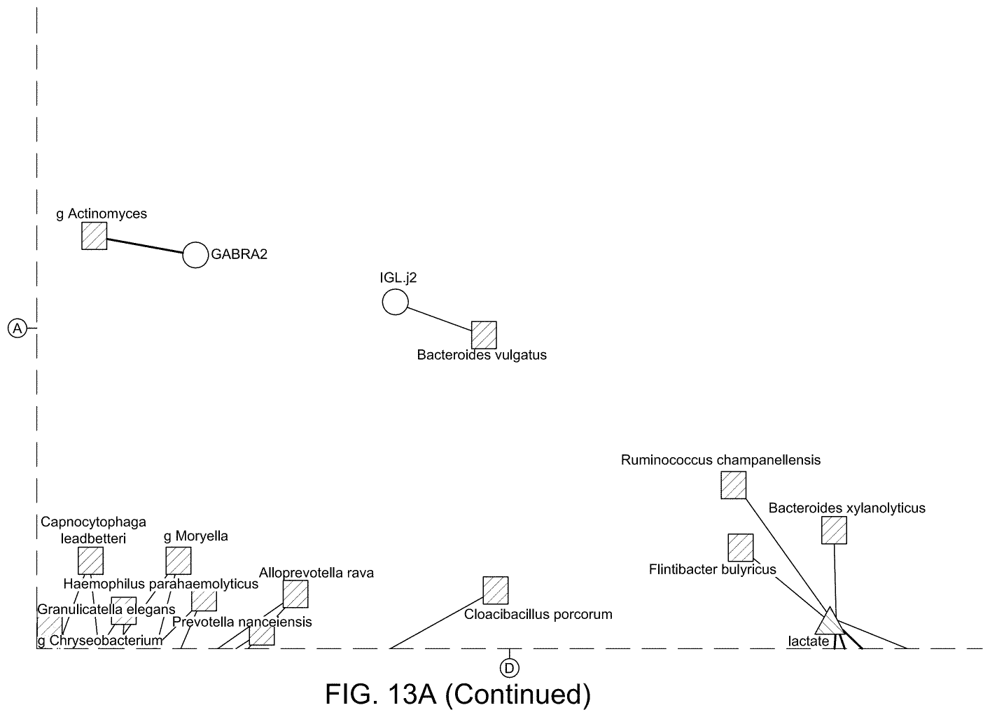

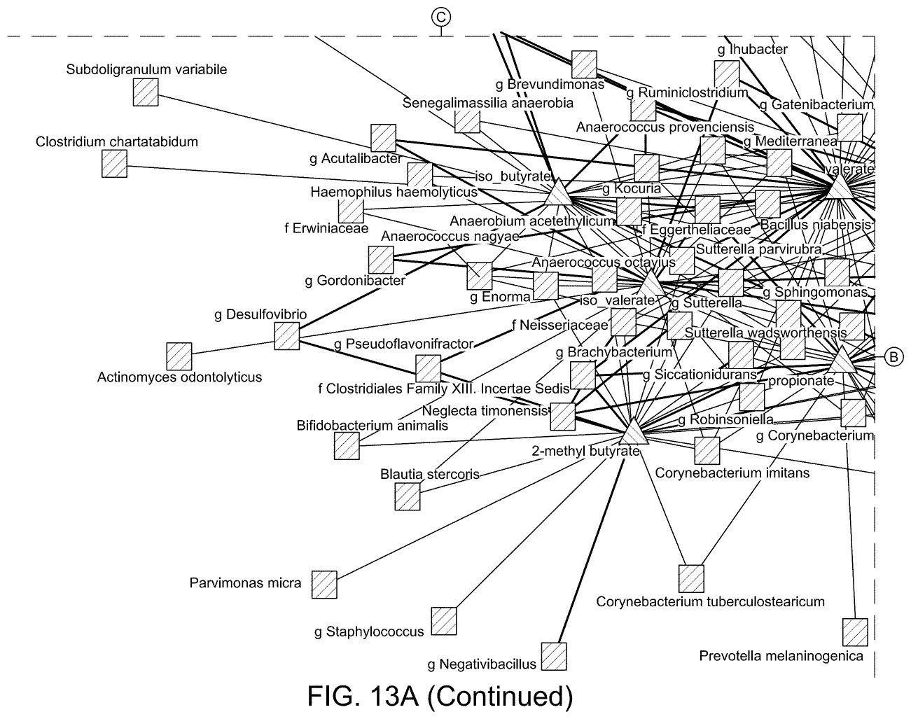

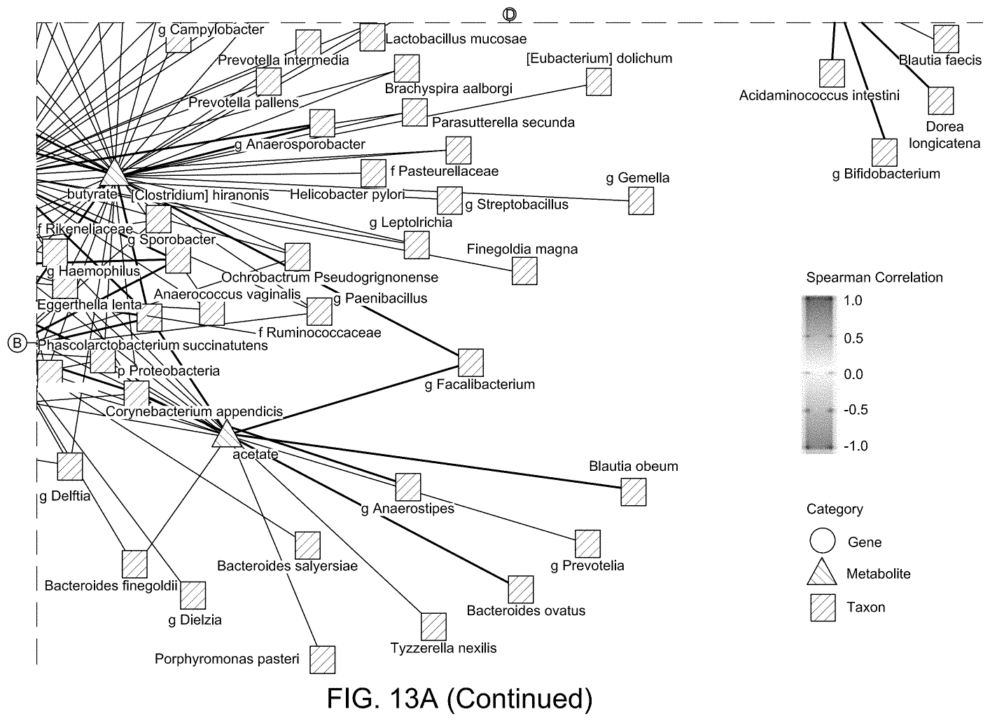

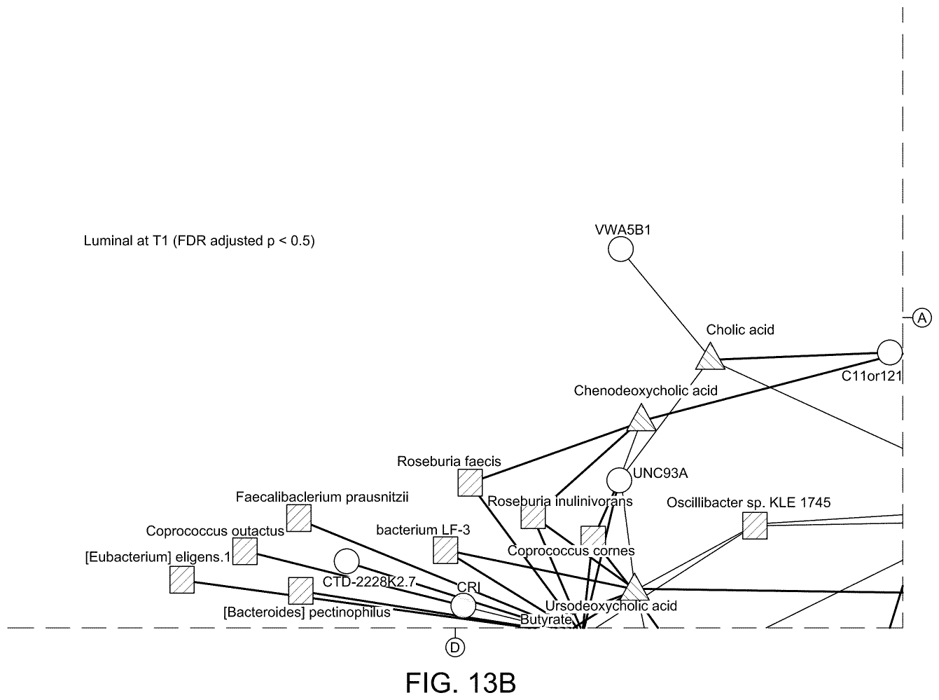

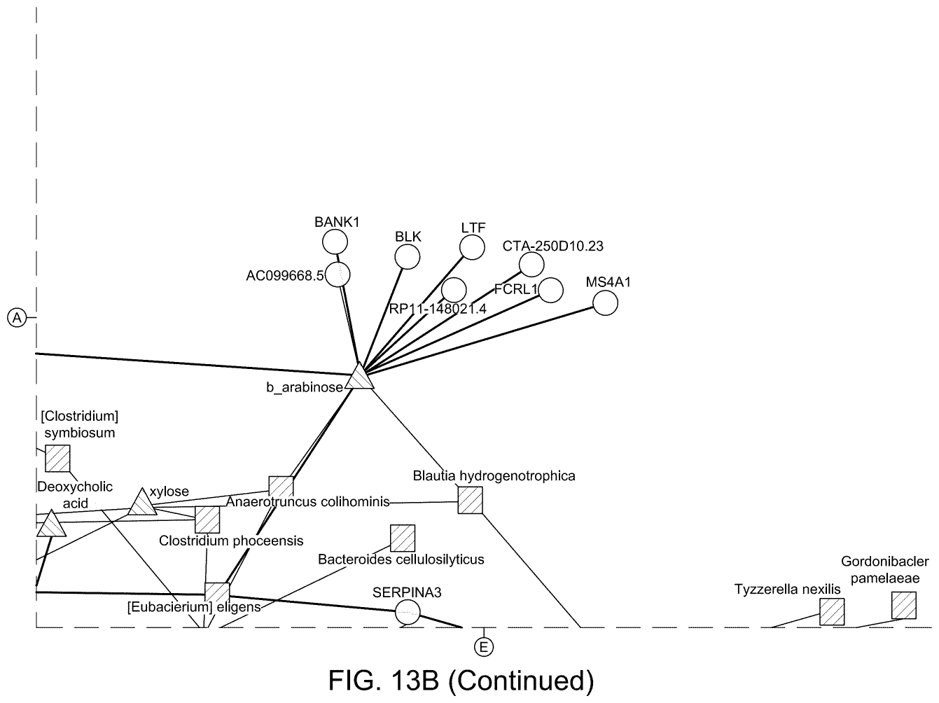

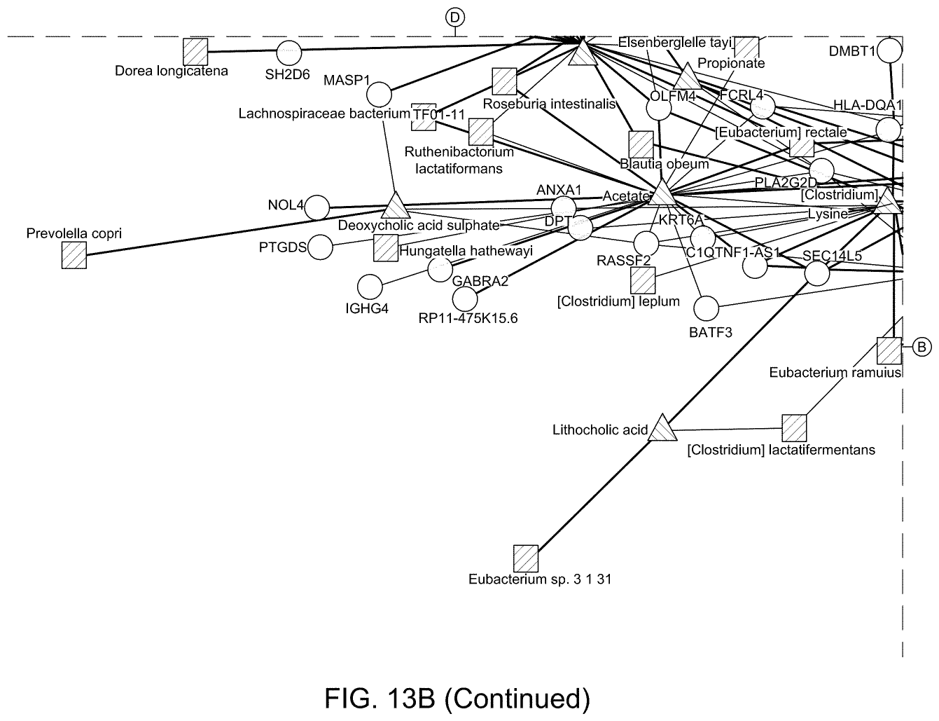

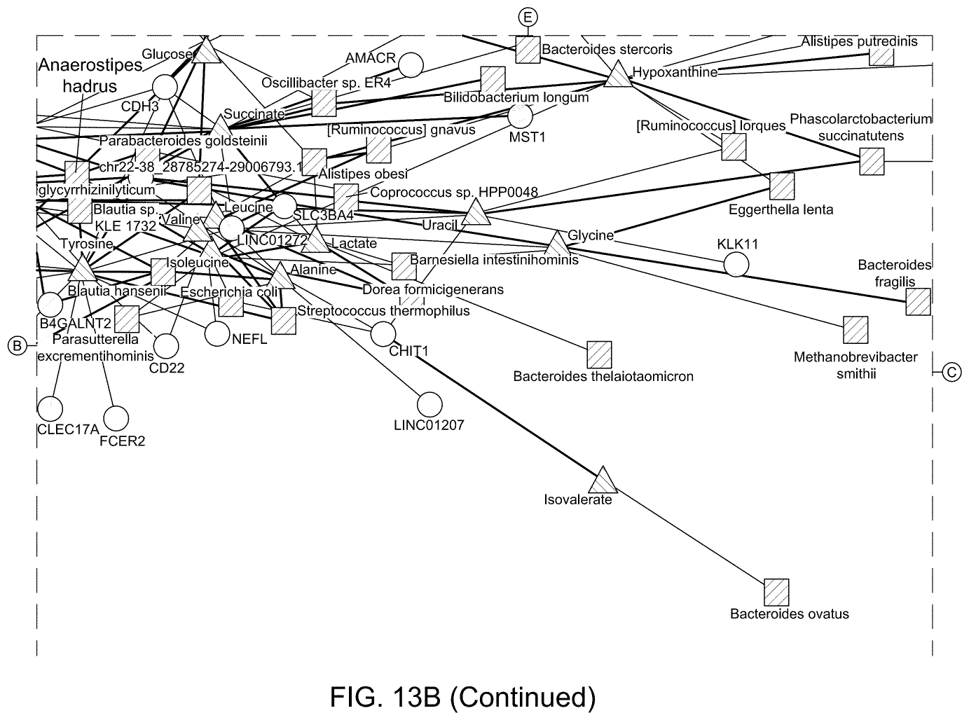

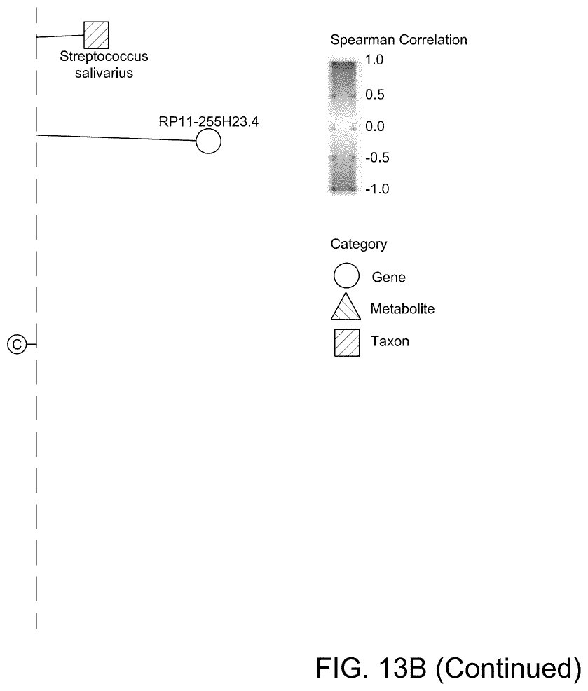

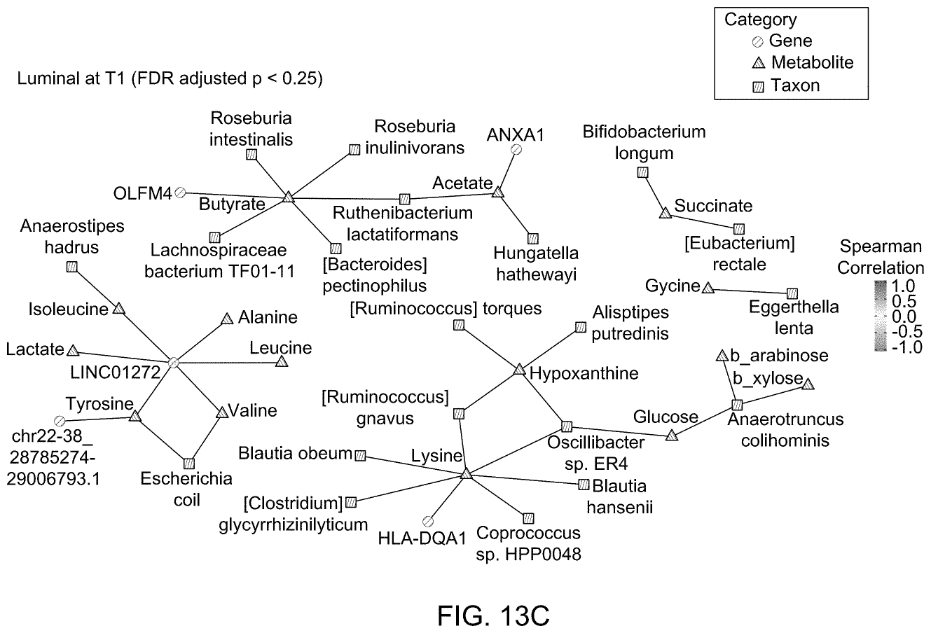

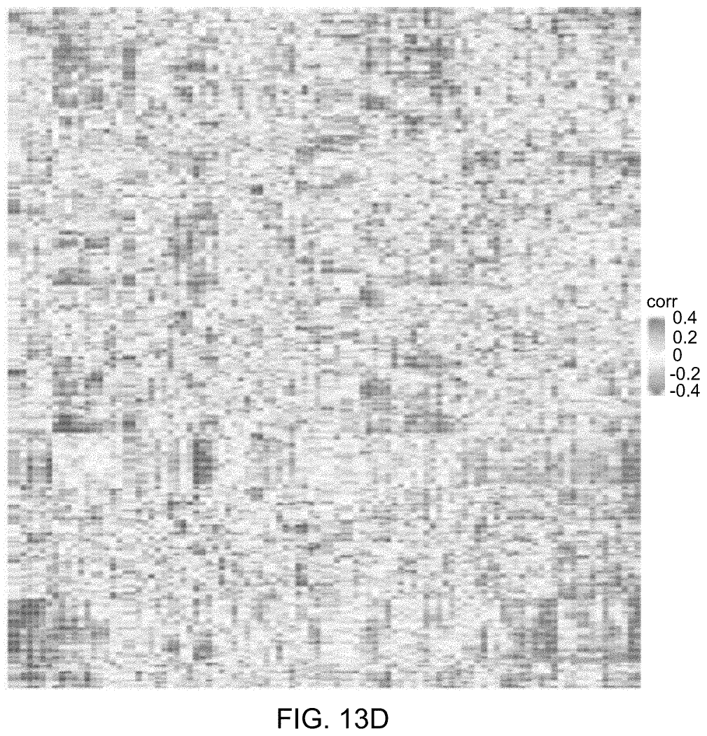

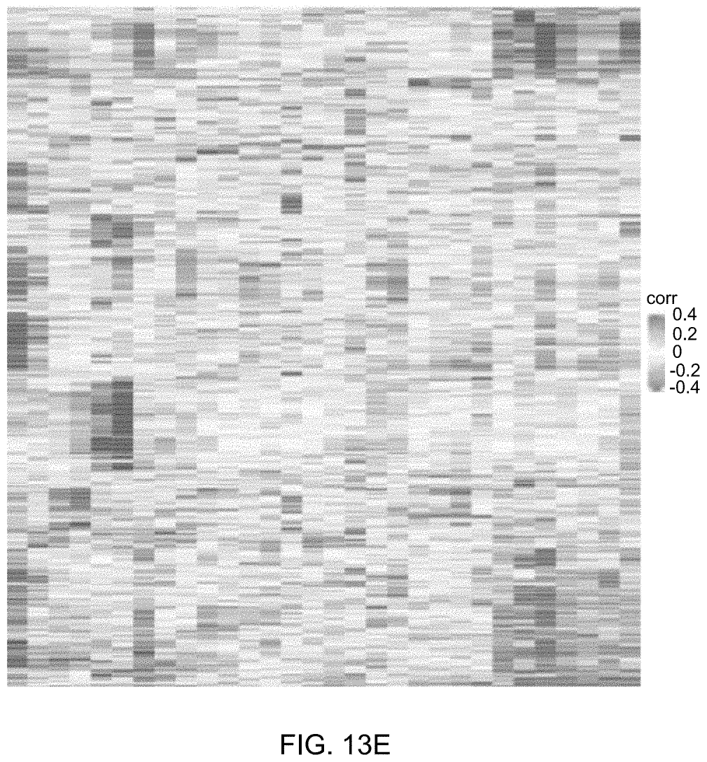

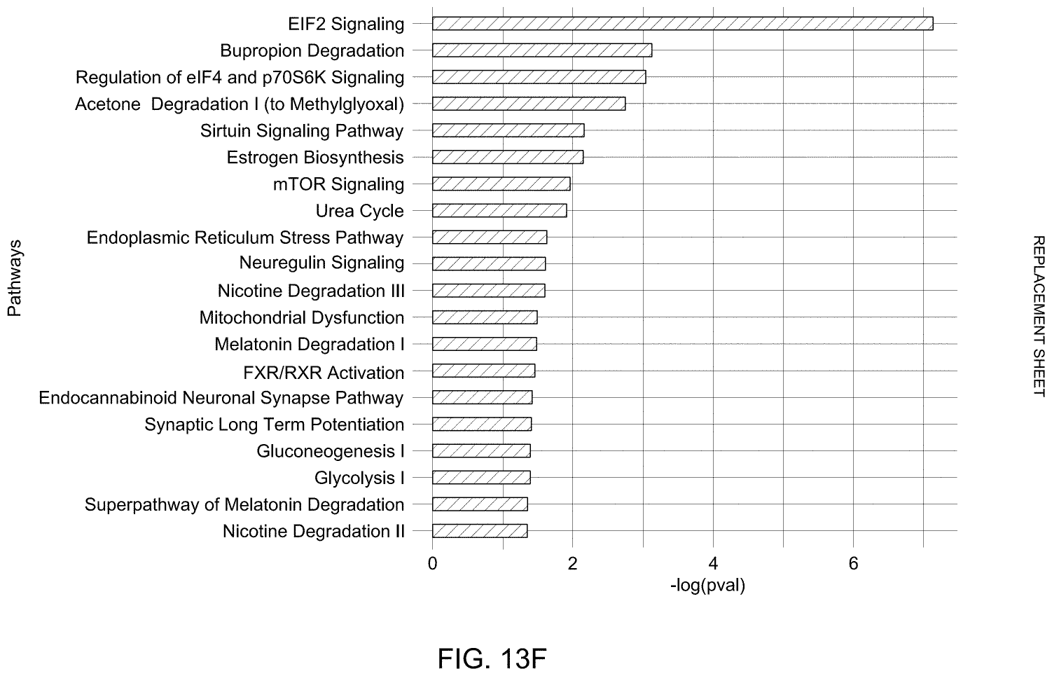

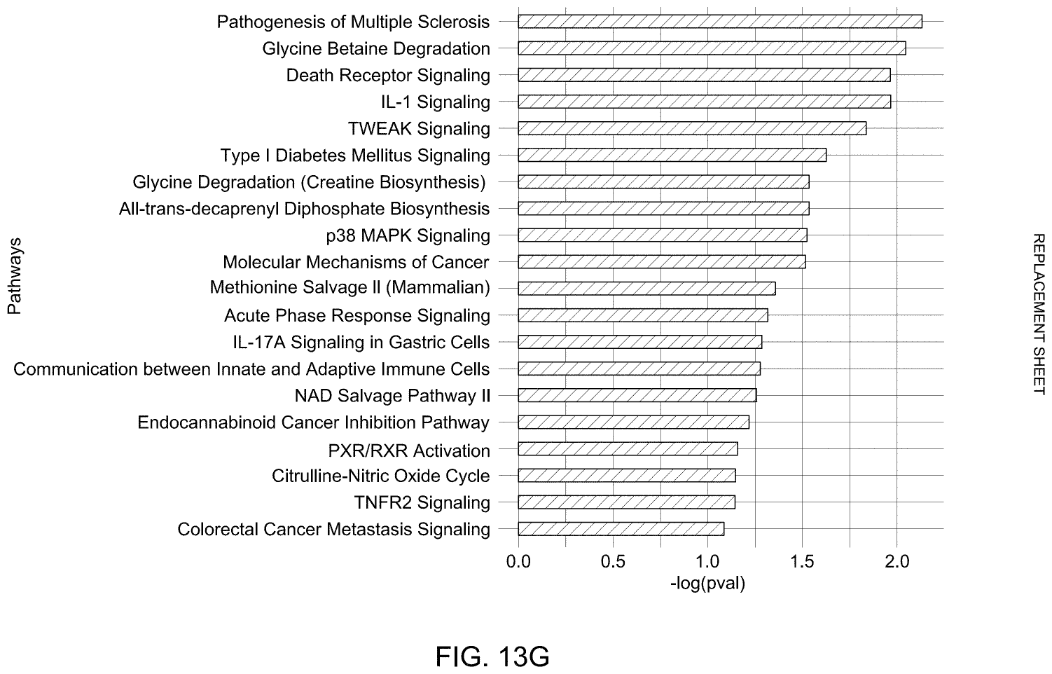

FIGS. 13A-13G: Data integration using correlation networks and Lasso regression. FIG. 13A is a diagram of a biopsy correlation network containing host transcriptome, biopsy metabolome, and biopsy microbiome. FIG. 13B is a diagram of a luminal correlation network containing host transcriptome, luminal metabolome and luminal microbiome. FIG. 13C is the same as FIG. 13B but at FDR cutoff<0.25. FIG. 13D is a heatmap representing the overall pattern of interaction between significant and stability-selected host genes (rows) and fecal metabolites (columns) identified by the lasso model at FDR<0.1 in IBS samples. FIG. 13E is the same as FIG. 13D, but for stability-selected host genes with microbial taxa. FIG. 13F is a bar graph showing the top 20 canonical enriched gene pathways associated with fecal metabolite levels for IBS samples. FIG. 13G is a bar graph showing the top 20 canonical enriched gene pathways associated to microbial taxa for IBS samples.

### DETAILED DESCRIPTION

This document provides materials and methods for improving gastrointestinal epithelial function and/or treating gastrointestinal disorders (e.g., functional gastrointestinal disorders). Functional gastrointestinal disorders are gastrointestinal disorders in which the bowel looks normal, but has abnormal function (pathophysiology) such as altered gut motility, secretion, and sensation. Examples of gastrointestinal disorders include, without limitation, functional gastrointestinal disorders (e.g., functional constipation), IBS, and inflammatory bowel diseases (e.g., infectious colitis, ulcerative colitis, Crohn's disease, ischemic colitis, radiation colitis, and microscopic colitis). In some cases, the materials and methods described herein can be used to supplement a mammal's diet with bacterial organisms and/or other agents having the ability to improve gastrointestinal functions. Examples of gastrointestinal functions include, without limitation, gastrointestinal motility, gastrointestinal secretion, and sensation.

As described herein, a longitudinal multi-omics study in IBS provided new understanding of the role of the gut microbiome in the pathophysiology of IBS. Extensive metadata and longitudinal host and microbial samples were collected from specific subsets of IBS patients. Multi-omic measurements, including microbial metagenome and metabolome, as well as host transcriptome and methylome, were integrated with host physiologic assessment. IBS subtype-specific mechanisms driven by altered microbial metabolism were identified that corresponded with concurrent changes in host physiology. In addition, using novel integration methods of multiple data layers, multiple unique pathways with potential relevance in IBS were identified, including a novel host-microbial co-metabolic purine pathway with biological implications in IBS.

This document therefore provides materials and methods for treating mammals identified as having gastrointestinal disorders such as IBS (e.g., IBS-C, IBS-D, or mixed IBS). For example, this document provides methods for reducing symptoms of IBS in a mammal by treating the mammal to increase the level of hypoxanthine in the mammal (e.g., in the gut of the mammal). In some cases, the methods provided herein can include administering one or more inhibitors of xanthine oxidase (e.g., a small molecule inhibitor of xanthine oxidase). In some cases, the level of hypoxanthine can be increased in a mammal by administering hypoxanthine in the diet, or by administering one or more bacterial organisms that promote hypoxanthine synthesis, one or more bacterial organisms that promote tryptamine synthesis (e.g., bacteria having tryptophan decarboxylase activity), one or more bacterial organisms having the ability to produce short chain fatty acids (SCFA), or one or more bacterial organisms having the ability to convert primary bile acids to secondary bile acids. In some cases, the level of hypoxanthine can be increased in a mammal by administering one or more agents that selectively remove bacteria that have xanthine oxidase activity, such that they can consume hypoxanthine.

Any appropriate mammal can be treated using the methods and materials provided herein. The mammal can be, for example, a human, a non-human primate, a cow, a horse, a pig, a sheep, a goat, a cat, a dog, a mouse, or a rat. Any suitable route of treatment can be used. For example, a pharmaceutical composition containing an agent that leads to increased hypoxanthine in a mammal can be administered locally (e.g., to the gut) or systemically. Administration can be, for example, oral, rectal, or parenteral (e.g., by subcutaneous, intrathecal, intraventricular, intramuscular, or intraperitoneal injection, or by intravenous drip), or topical (e.g., transdermal, sublingual, ophthalmic, or intranasal), or can occur by a combination of such methods. Administration can be rapid (e.g., by injection) or can occur over a period of time (e.g., by slow infusion or administration of a slow release formulation). In some cases, the treatment can be administered such that its delivery is restricted to the GI tract (e.g., oral or rectal delivery).

In some cases, the level of hypoxanthine can be increased in a mammal by administering hypoxanthine to the mammal. For example, hypoxanthine can be administered orally (e.g., in tablet, capsule, or liquid form, or in a food product) or rectally (e.g., as a suppository or an enema) at a dose of about 0.1 to about 1000 mg/kg body weight per day (e.g., about 0.1 to about 1, about 1 to about 10, about 10 to about 25, about 25 to about 100, about 25 to about 50, about 50 to about 100, about 100 to about 250, about 250 to about 500, or about 500 to about 1000 mg/kg per day.

In some cases, the methods provided herein can include administering one or more agents that inhibit metabolism of hypoxanthine (e.g., xanthine oxidase inhibitors). Non-limiting examples of xanthine oxidase inhibitors include purine analogues such as allopurinol, oxypurinol, tisopurine, febuxostat, topiroxostat, and inositols. Other examples of xanthine oxidase inhibitors that may be used in the methods provided herein include flavonoids such as kaempferol, myricetin, and quercetin, planar flavones and flavonols having a 7-hydroxyl group, Cinnamomum osmophloeum oil, propolis, and Pistacia integerrima extract. Agents that inhibit hypoxanthine metabolism can be administered orally (e.g., in tablet, capsule, or liquid form, or in a food product) or rectally (e.g., as a suppository or an enema) in at a dose of about 0.1 to about 1000 mg/kg per day (e.g., about 0.1 to about 1, about 1 to about 10, about 10 to about 25, about 25 to about 100, about 25 to about 50, about 50 to about 100, about 100 to about 250, about 250 to about 500, or about 500 to about 1000 mg/kg per day). In some cases, the methods provided herein can include administering a composition containing at least one type of bacteria (e.g., intestinal bacteria) with a desired activity (e.g., the ability to produce hypoxanthine). An “intestinal bacteria” is any bacterial species that normally lives in the digestive tracts of a mammal. Examples of intestinal bacteria that can be used as described herein include, without limitation, organisms belonging to the genera Prevotella, Bacteroides, Clostridium, Faecalibacterium, Eubacterium, Ruminococcus, Peptococcus, Peptostreptococcus, Bifidobacterium, Escherichia, Lactobacillus, Akkermansia, Roseburia, Enterococcus, Bacillus, Bacteroides, Lachnospiraceae, Hungatella, Anaerostipes, and Blautia. In some cases, a fungal composition containing a fungal organism (e.g., intestinal fungus) having a particular activity (e.g., tryptophan decarboxylase activity) can be used in place of a bacterial composition or in addition to a bacterial composition in the methods provided herein. Examples of intestinal fungi that can be used as described herein include, without limitation, Candida, Saccharomyces, Aspergillus, and Penicillium.

The methods provided herein can include, for example, administering at least one live bacterial organism that has the ability to produce hypoxanthine. Examples of bacterial organisms that can produce hypoxanthine include, without limitation, Escherichia coli K12, Enterococcus spp., Faecalibacterium spp., Bacillus spp., and Bacteroides thetaiotaomicron engineered to produce hypoxanthine. For example, B. thetaiotaomicron can be genetically engineered to contain and express one or more genes such as those encoded by the pur operon in E. coli or other strains that encode hypoxanthine producing enzymes. Enzymes involved in hypoxanthine synthesis include, without limitation, adenine deaminase.

In some cases, the methods provided herein can include administering at least one live bacterial organism that promotes tryptamine synthesis (e.g., one or more bacterial organisms having tryptophan decarboxylase activity). Examples of bacterial organisms that can promote tryptamine synthesis include, without limitation, Prevotella spp., Bacteroides spp., Clostridium spp. (e.g., C. sporogenes), Faecalibacterium spp., Eubacterium spp., Ruminococcus spp. (e.g., R. gnavus), Peptococcus spp., Peptostreptococcus spp., Bifidobacterium spp., Escherichia spp., Lactobacillus spp., Akkermansia spp., and Roseburia spp. Compositions containing bacterial organisms that can promote tryptamine synthesis are further described elsewhere (U.S. Patent Application Publication No. 2017/0042860).

In some cases, the methods provided herein can include administering at least one live bacterial organism having the ability to produce SCFA (e.g., acetate or butyrate). Examples of bacteria strains that have the ability to produce SCFA include Faecalibacterium prausnitzii (clostridial cluster IV), Anaerostipes spp., Eubacterium spp., Roseburia spp. (clostridial cluster XIVa), Blautia spp., Bifidobacteria spp., Lactobacillus spp., Akkermansia muciniphila, Prevotella spp., and Ruminococcus spp. The bacteria can produce short chain fatty acids due to the activity of one or more enzymes in a particular pathway, depending on the substrate. For example, a bacterial organism having the ability to produce SCFA can have 3-hydroxybutyryl-CoA dehydratase activity, 2-hydroxyglutarate dehydrogenase activity; glutaconate CoA transferase activity (α, β subunits), 2-hydroxy-glutaryl-CoA dehydrogenase activity (α, β, γ subunits), glutaconyl-CoA decarboxylase activity (α, β subunits), thiolase activity, β-hydroxybutyryl-CoA dehydrogenase activity, crotonase activity, butyryl-CoA dehydrogenase activity (including electron transfer protein α, β subunits), lysine-2,3-aminomutase activity, β-lysine-5,6-aminomutase activity (α, β subunits, 3,5-diaminohexanoate dehydrogenase activity, 3-keto-5-aminohexanoate cleavage enzyme activity, 3-aminobutyryl-CoA ammonia lyase activity, 4-hydroxybutyrate dehydrogenase activity, 4-hydroxybutyryl-CoA dehydratase activity, vinylacetyl-CoA 3,2-isomerase activity, butyryl-CoA:4-hydroxybutyrate CoA transferase activity, butyryl-CoA:acetate CoA transferase activity, butyryl-CoA:acetoacetate CoA transferase activity (α, β subunits), phosphate butyryltransferase activity, butyrate kinase activity, or any combination thereof.

In some cases, the methods provided herein can include administering at least one live bacterial organism having the ability to convert primary bile acids to secondary bile acids. For example, a bacterial organism can be administered that has the ability to convert cholic acid to deoxycholic acid and/or the ability to convert chenodeoxycholic acid to lithocholic acid. Suitable bacterial organisms include, without limitation, Clostridium spp. (e.g., Clostridium scindens or an engineered Clostridia sp.). Such bacteria can have the ability to convert primary bile acids to secondary bile acids due to the activity of a series of enzymes that are part of the 7α-dehydroxylation pathway.

Compositions containing at least one bacterial strain as described herein also can contain one or more additional probiotic microorganisms. Examples of other probiotic microorganisms that can be included within a composition containing at least one bacterial strain having a desired activity as described herein include, without limitation, Prevotella coprii, Bifidobacterium infantis, Lactobacillus rhamnosis GG, Lactobacillus plantarum, Bifidobacterium breve, Bifidobacterium longum, Lactobacillus acidophilus, Lactobacillus paracasei, Lactobacillus bulgaricus, Streptococcus thermophilus, and Faecalibacterium prauznitzii.

In some cases, bacteria can be engineered to have a desired activity (e.g., tryptophan decarboxylase activity). For example, bacteria can be engineered to express an exogenous nucleic acid encoding a polypeptide having tryptophan decarboxylase activity. Bacteria engineered to have tryptophan decarboxylase activity can include an exogenous nucleic acid encoding a polypeptide having tryptophan decarboxylase activity derived from any appropriate source. Examples of bacteria that can be engineered to express a polypeptide having tryptophan decarboxylase activity include, without limitation, Escherichia coli and Bacteroides thetaiotaomicron. Examples of nucleotide sequences that encode a tryptophan decarboxylase include, without limitation, those nucleic acid sequence that encode the amino acid sequence set forth in GENBANK® Accession No. ZP_02040762 (GI No. 154503702). Any appropriate method can be used to engineer bacteria to express an exogenous nucleic acid encoding a polypeptide having tryptophan decarboxylase activity. In some cases, a promoter sequence can be operably linked to a nucleic acid sequence that encodes a polypeptide having tryptophan decarboxylase activity to drive expression of the tryptophan decarboxylase. An example of such a promoter sequence includes, without limitation, a CMV promoter. In some cases, a bacterial strain having trp decarboxylase activity can be engineered to have enhanced tryptophan production.

Compositions used in the methods provided herein can include any amount of bacteria having a desired activity (e.g., tryptophan decarboxylase activity). In some cases, a composition provided herein can contain bacteria having a desired activity in an amount such that from about 0.001 to about 100 percent (e.g., from about 1 percent to about 95 percent, from about 10 to about 95 percent, from about 25 to about 95 percent, from about 50 to about 95 percent, from about 20 to about 80 percent, from about 50 to about 95 percent, from about 60 to about 95 percent, from about 70 to about 95 percent, from about 80 to about 95 percent, from about 90 to about 95 percent, from about 95 to about 99 percent, from about 50 to about 100 percent, from about 60 to about 100 percent, from about 70 to about 100 percent, from about 80 to about 100 percent, from about 90 to about 100 percent, or from about 95 to about 100 percent), by weight, of the composition can be bacteria having the desired activity.

Any amount of a composition containing at least one bacterial strain having a desired activity can be administered to a mammal. The dosages of the compositions provided herein can depend on many factors, including the desired results. Typically, the amount of bacteria contained within a single dose can be an amount that effectively exhibits improved gastrointestinal function within the mammal. For example, a composition containing at least one bacterial strain can be formulated in a dose such that a mammal receives from about 10³ to about 10¹² (e.g., about 10³ to about 10⁵, about 10⁵ to about 10⁷, about 10⁷ to about 10⁹, or about 10⁹ to about 10¹²) bacteria having the desired activity.

In some cases, a composition used in the methods provided herein can contain bacteria having a desired activity in the amounts and dosages as described elsewhere for probiotic bacteria (U.S. Patent Application Publication No. 2008/0241226; see, e.g., paragraphs [0049-0103]). In addition, a composition provided herein containing bacteria can be administered as described elsewhere for probiotic bacteria (U.S. Patent Application Publication No. 2008/0241226; see, e.g., paragraphs [0049-0103]).

In some cases, a composition used in the methods described herein can contain at least one agent or bacterial strain and can be in the form of an oral medicament or nutritional supplement, or in the form of a medicament for rectal administration. For example, compositions for oral administration can be in the form of a pill, tablet, powder, liquid, or capsule. Tablets or capsules can be prepared with pharmaceutically acceptable excipients such as binding agents, fillers, lubricants, disintegrants, or wetting agents. In some cases, tablets can be coated. In some cases, a composition containing at least one bacterial strain can be formulated such that the bacteria are encapsulated for release within the intestines of a mammal. Liquid preparations for oral administration can take the form of, for example, solutions, syrups, or suspension, or they can be presented as a dry product for constitution with saline or other suitable liquid vehicle before use. In some cases, a composition provided herein containing at least one bacterial strain can be in a dosage form as described elsewhere (U.S. Patent Application Publication No. 2008/0241226; see, e.g., paragraphs [0129-0135]). For example, a composition provided herein can be in the form of a food product formulated to contain at least one bacterial strain having a desired activity. Examples of such food products include, without limitation, milk (e.g., acidified milk), yogurt, milk powder, tea, juice, beverages, candies, chocolates, chewable bars, cookies, wafers, crackers, cereals, treats, and combinations thereof. A composition for rectal administration can be in the form of a suppository, or an enema, for example.

In some cases, a composition containing at least one bacterial strain also can contain a pharmaceutically acceptable carrier for administration to a mammal, including, without limitation, sterile aqueous or non-aqueous solutions, suspensions, and emulsions. Examples of non-aqueous solvents include, without limitation, propylene glycol, polyethylene glycol, vegetable oils, and organic esters. Aqueous carriers include, without limitation, water, alcohol, saline, and buffered solutions. Pharmaceutically acceptable carriers also can include physiologically acceptable aqueous vehicles (e.g., physiological saline) or other known carriers for oral or rectal administration.

In some cases, methods provided herein can include selectively removing from a mammal one or more bacterial organisms having the ability to consume hypoxanthine. Examples of bacteria that can consume hypoxanthine include, without limitation, Lachnospiraceae spp. and Hungatella hathewayi. Methods for selectively removing such bacteria can include administering an agent such as a bacteriophage (e.g., a naturally occurring or engineered bacteriophage that can recognize and kill bacteria that consume hypoxanthine), or an antimicrobial compound (e.g., a lantibiotic).

In general, a treatment that increases hypoxanthine levels in a mammal having IBS can reduce one or more symptoms of IBS in the mammal. For example, administration of an agent that results in increased hypoxanthine levels can reduce abdominal pain or discomfort, improve stool form, and/or regulate stool frequency in the mammal. After identifying a mammal as having a need for IBS treatment, the mammal can be treated with a composition containing a hypoxanthine-increasing agent. The composition can be administered to the mammal in any amount, at any frequency, and for any duration effective to achieve a desired outcome (e.g., to reduce one or more symptoms of IBS) in the mammal. In some cases, for example, a composition can be administered to a mammal repeatedly (e.g., once or more than once a day, once or more than once a week, or once or more than once a month). For example, a composition containing a hypoxanthine-increasing agent can be administered daily to treat symptoms and/or to prevent or reduce the likelihood of IBS flares. The frequency of administration can remain constant or can be variable during the duration of treatment. Various factors can influence the frequency of administration. For example, the effective amount, duration of treatment, route of administration, and severity of condition may require an increase or decrease in administration frequency.

In some cases, particular forms of IBS (e.g., IBS-C or IBS-D) can be treated in a mammal by administering one or more agents targeted to those disorders. For example, a mammal identified as having IBS-C can be treated with an agent that increases tryptamine or SCFA production by gut bacteria in the mammal. In another example, a mammal identified as having IBS-D can be treated with an agent that increases bile acid biotransformation in the mammal.

This document also provides methods for identifying a mammal as having IBS. The methods can include measuring the level of hypoxanthine in a biological sample (e.g., a stool sample or a colonic mucosa sample) from a mammal, and the mammal can be identified as having IBS when the measured level of hypoxanthine is less than a control level of hypoxanthine. The control level can be, for example, a level measured in a corresponding biological sample from a mammal or a population of mammals that do not have IBS. A level that is “less than” a control level is a level that is at least 5% lower than the control level (e.g., at least 10%, at least 20%, at least 25%, at least 30%, or at least 50% lower than the control level). Any appropriate method can be used to determine the level of hypoxanthine in a sample. Suitable methods include, without limitation, ¹H-NMR, LC-MS, and xanthine/xanthine oxidase assays.

In addition, this document provides methods for identifying a mammal as having IBS-C or IBS-D, or as being likely to have IBS-C or IBS-D. For example, the methods can include measuring the level of tryptamine or SCFA in a biological sample (e.g., a stool sample or a colonic mucosa sample) from a mammal, and the mammal can be identified as having (or being likely to have) IBS-C when the measured level of tryptamine or SCFA is less than a control level of tryptamine or SCFA. In some cases, the methods can include measuring the level of primary bile acids in a biological sample from a mammal, and identifying the mammal as having (or being likely to have) IBS-D when the measured level of primary bile acids is less than a control level of primary bile acids. Again, the control level can be, for example, a level measured in a corresponding biological sample from a mammal or a population of mammals that do not have IBS-C or IBS-D. A level that is “less than” a control level is a level that is at least 5% lower than the control level (e.g., at least 10%, at least 20%, at least 25%, at least 30%, or at least 50% lower than the control level). Any appropriate method can be used to determine the level of tryptamine, SCFA, or primary bile acids in a sample. Suitable methods include, without limitation, GC-MS/MS and LC-MS.

The invention will be further described in the following examples, which do not limit the scope of the invention described in the claims.

### EXAMPLES

### Example 1

### Methods

Recruitment and exclusion criteria: Participants were recruited through Mayo Clinic Institutional Review Board approved advertisements. Healthy subjects and IBS-C and IBS-D patients between 18-65 years old who expressed interest were invited to undergo screening to assess eligibility. Participants were given the option of undergoing two flexible sigmoidoscopies. All IBS-C and IBS-D subjects fulfilled Rome III criteria. Recruitment of healthy subjects was matched with IBS subjects for age, sex and BMI. Volunteers with prior history of abdominal surgeries (except appendectomy and cholecystectomy), diagnosis of inflammatory bowel disease, microscopic colitis, celiac disease, or other inflammatory conditions, antibiotic use within the past 4 weeks, bleeding risk or taking medication that increases bleeding risk (only for those who chose to undergo the flexible sigmoidoscopies), bowel prep for colonoscopy in the past week, pregnancy, plan to become pregnant during study, being a vulnerable adult, and age below 18 or over 65 were excluded. In addition, people were excluded if they had other diseases, conditions, or habits that would interfere with study completion, increased risks with flexible sigmoidoscopies (if chosen), or that in judgment of investigator would potentially interfere with compliance to study or adversely affect outcomes.

Specimen collection and data generation: Stool specimens were completed via home collection kits at the earliest convenience after the initial visit and then monthly for six months. Sample tubes were returned with frozen gel packs overnight using FedEx or dropped off at the clinical core facility of the Mayo Clinic Center for Cell Signaling, where samples were stored at −80° C. Blood samples (plasma, serum, whole) were collected at the initial visit only and stored at −80° C. upon further processing. Biopsies were obtained through flexible sigmoidoscopy from the sigmoid colon 20-30 cm from the anal verge essentially as described elsewhere (Bhattarai et al., Cell Host Microbe 2018, 23:775-785 e775; and Peters et al., J Gastroenterol 2017, 112:913-923). Up to two tap water enemas were given to cleanse the colon for each procedure. All endoscopic procedures were performed by a single endoscopist, and up to twelve colonic biopsies were collected using a large-capacity (2.8 mm) biopsy forceps without pin. Depending on downstream processing, the biopsies were placed in RNAlater stabilization solution (Life Technologies), directly frozen in liquid nitrogen, or placed in glucose Krebs solution on ice (composition in mM: 11.5 D-glucose, 120.3 NaCl, 15.5 NaHCO₃, 5.9 KCl, 1.2 NaH₂PO₄, 2.5 CaCl₂.2H₂O, and 1.2 MgCl₂; pH 7.3-7.4) and immediately transported to the laboratory for experiments.

Participant and sample metadata: Additional info on study subjects was collected at the first visit after study consent for IBS and healthy volunteers. This included recording of medical history and a limited exam by study physician where height, weight, BMI and vital signs were noted. Further, study subjects underwent a dietitian consult where explanation on Food Frequency Questionnaires (FFQ) and 24-hour dietary recall questionnaire training was given. Additional questionnaires at the first visit were Rome III criteria for IBS diagnosis, IBS symptom severity (also completed monthly for IBS participants), microbiome health, bowel disease questionnaire (BDQ-6), Hospital Anxiety and Depression, IBS Quality of Life, and 7-day Bowel Diary (also completed monthly for all participants).

Ussing chamber experiments: Colonic mucosal secretory responses were assessed using Ussing chamber setups. Biopsies were mounted within 45 minute of collection in 4 ml Ussing chambers (Physiologic Instruments; San Diego, Calif.) with an aperture of 0.31 cm². The basolateral side of the chamber was bathed with 4 mL of glucose Krebs solution while the apical side was bathed with 4 mL of Krebs Mannitol solution (composition in mM: 767 11.5 D-mannitol, 120.3 NaCl, 15.5 NaHCO₃, 5.9 KCl, 1.2 NaH₂PO₄, 2.5 CaCl₂.2H₂O, and 1.2 MgCl₂; pH 7.3-7.4). The chamber was bubbled with a 97% O₂ and 3% CO₂ gas mixture. Tissue viability was confirmed by using concentration response measurements to acetylcholine (1 mM-3 mM) added on the submucosal side prior to the start of experiments. Short circuit current (Isc) was continuously recorded using Acquire and Analyze software (Physiologic Instruments). ΔIsc values were calculated using Isc measurements before and after application of compounds to the basolateral side and normalized to the tissue area. Tryptamine and serotonin were added at 11 cumulatively increasing concentrations from 0.003 μM to 300 μM. Imax is the maximal Isc value achieved at any of the concentrations.

Microbiome DNA sequencing and alignment: DNA extraction and sequencing was performed at the University of Minnesota Genomics Center (UMGC). DNA was extracted from stool and biopsy sections using the Qiagen PowerSoil kit (Qiagen; Germantown, Md.), and was quantified using a NanoDrop-8000 UV-Vis Spectrophotometer (Thermo Scientific; Wilmington, Del.) and PicoGreen assays. Shotgun metagenomic sequencing library preparation for stool samples was completed using a modified NexteraXT protocol followed by sequencing on a HiSeq 2500 (Rapid Mode) with 100 bp single-end reads (1×100) or on a NextSeq with 150 bp single-end reads (1×150). Shotgun reads were trimmed to a maximum of 100 bp prior to alignment. Shotgun sequences were aligned to the RefSeq representative prokaryotic genome collection (release 86) at 97% identity with BURST using default settings (Al-Ghalith et al., doi.org/10.5281/zenodo.806850). The generated alignment table was filtered by dropping samples with low depth (<10,000 reads per sample). Functional profiling of the shotgun sequencing data was completed using the KEGG Orthology group annotations for RefSeq-derived genes from direct alignment. KEGG Orthology profiles were also predicted from reference genomes and the predicted profiles were augmented to improve the estimates of low-abundance genes using SHOGUN (github.com/knights-lab/SHOGUN). Biopsy samples were sequenced via amplification of the V4 region of the 16S ribosomal RNA gene (Gohl et al., Nat Biotechnol 2016, 34:942-949), followed by paired-end 2×250 bp sequencing on an Illumina MiSeq. Adapters were trimmed and low-quality reads (<25 Q-score) were dropped using Shi7 (Al-Ghalith et al., mSystems 2018, 3(3):e00202-17). Amplicon reads were stitched also using Shi7 (Al-Ghalith et al., supra). Amplicon sequences were aligned to the 16S rRNA genes from the same bacterial genomes in the shotgun sequencing approach using BURST (Al-Ghalith et al., doi.org/10.5281/zenodo.806850) with the same setting as above.

Microbiome data analysis: Downstream analysis of taxa and KEGG Orthology tables was performed in R (R Foundation for Statistical Computing, Vienna, Austria). Computing PERMANOVA, Shannon diversity, and Bray Curtis dissimilarity was done using adonis, diversity(x, index=“shannon”), and vegdist (x, method=“bray”) functions from the vegan package. Before testing for taxa differences between the subgroups, taxa were removed that were absent in 90% of the subjects (averaged data excluding flares). To identify differentially abundant features an FDR cutoff of <0.25 was used. In specified cases, this cutoff was made more rigorous post-hoc to display only top features due to the great number of significant changes at FDR<0.25. Bray-Curtis dissimilarity (BCD)-based irregularity (BCDI) was computed by extracting the pairwise dissimilarities between all healthy control (HC) and HC or IBS samples, and the median of these dissimilarities was stored. The 90th percentile of the HC values was used as a cutoff for identifying microbiome samples that were different compared to those of HC. For this analysis samples from one HC subject (10007557) were removed since the median of these samples was above the 90th percentile of the HC BCDI scores. A sensitivity analysis of the 90th percentile cutoff values was performed by randomly drawing one sample per HC subject and identifying the BCDI within these samples 500 times. In addition, the 90th percentile cutoffs from averaged HC microbiome abundances were computed. Taking the average did not change the 90th percentile cutoff (0.63).

Metabolomics—¹H NMR untargeted metabolome profiling of serum and stool samples: Aliquoted stool samples (˜100 mg) were randomized in order and transferred to a screw-cap tube containing 50 mg 1.0 mm Zirconia beads (BioSpec). Metabolites were extracted by addition of 400 μL of acetonitrile:H2O (approximate volumetric ratio of 1:3) and homogenized for 30 seconds in a Biospec beat beater at maximum speed. The homogenized samples were then centrifuged for 20 minutes at 16000×g, after which the supernatant was transferred to Spin-X 0.22 μm spin filter tubes (COSTAR®) and centrifuged for 30 minutes at 16000×g. 80 μL of the filtered samples was aliquoted into 96 well plates, and 10 μL was kept separately for downstream quality control purposes. Samples were dried under nitrogen flow before reconstituting in 540 μL of D2O and 60 μL of NMR buffer, all in 96 well deep well plates (COSTAR®). The plate was then placed on an Eppendorf MixMate plate shaker at 1300 rpm for 5 minutes. The reconstituted fecal water and buffer mixture was transferred to 5 mm NMR tubes. Plasma buffer with 1.5 M KH₂PO₄ was prepared by dissolving 20.4 g of KH2PO4 in 80 mL of D₂O. 6 mL of D₂O containing 100 mg of 3-(trimethylsilyl) propionic-2,2,3,3-d4 acid sodium salt (TSP) (Millipore-Sigma) and 13 mg of NaN₃ was added and mixed by shaking and sonication. The pH was adjusted to pH 7.4 with NaOH pellets. The total volume was adjusted with D₂O.

Serum samples were thawed and centrifuged at 4° C. at 12000×g for 5 minutes. All samples were kept at −40° C. or colder until analysis. 90 μL of the supernatant was mixed with 90 μL of plasma buffer before being transferred to a 3 mm NMR tube. Plasma buffer with 0.075 M NaH₂PO₄ was prepared by dissolving 1.064 g of NaH₂PO₄ in 80 mL of D₂O. 4 mL of D₂O containing 80 mg of 3-(trimethylsilyl) 850 propionic-2,2,3,3-d4 acid sodium salt (TSP) (Millipore-Sigma) and 40 mg of NaN3 was added and mixed by shaking and sonication. The pH was adjusted to pH 7.4 with NaOH pellets. Total volume was adjusted with D₂O.

Metabolic profiles were recorded essentially as described elsewhere (Dona et al., Anal Chem 2014, 86:9887-9894) on a Bruker 600 MHz spectrometer (Bruker Biospin) set at a constant temperature of 300K for fecal samples and 310K for plasma samples. A 1D nuclear Overhauser enhancement spectroscopy (NOSEY) experiment and a 2D J-resolved experiment was performed for each fecal and serum sample. A total of 32 scans were acquired with an acquisition time of 4 minutes and 3 seconds per fecal sample following 4 dummy scans and the spectral data was collected into 64K data points. Automatic phasing, baseline correction and spectral calibration to TSP (0 ppm) was performed in Topspin 3.1 (Bruker Biospin).

The pre-processed spectral data was imported into MATLAB (Version 8.3.0.532 864 R2014a, Mathworks Inc, Natick, Mass., USA). A series of in-house scripts were used for the following executions. The spectra were manually aligned to correct for subtle alterations in the chemical shifts of the peaks due to variation in pH. To account for the difference in sample concentration, probabilistic quotient normalization (PQN) was applied to the spectral data. A projection to latent structures-discriminant analysis (PLS-DA) model based on the Monte Carlo cross-validation (MCCV) method was constructed on the complete spectral profiles to identify discriminatory features in relevant comparisons (Garcia-Perez et al., Lancet Diabetes Endocrinol 2017, 5:184-195; and Posma et al., J Proteome Res 2018, 17:1586-1595). A total of 1,000 MCCV models with 25 bootstrap rounds in each model was used to assess model robustness and to calculate the mean prediction (Tpred) of each sample. Discriminatory 873 spectral features were annotated using statistical total correlation spectroscopy (STOCSY) (Cloarec et al., Anal Chem 2005 77:1282-1289) and a combination of in-house and online databases (www.hmdb.ca). An in-house developed peak integration script was applied to calculate the integral of spectral peaks of interest.

Metabolomics—Bile acid profiling through LC-MS/MS: Metabolites were extracted as detailed above for ¹H NMR. Samples were analyzed on an ACQUITY ultraperformance liquid-chromatography (UPLC) system (Waters Ltd., UK) coupled to a Xevo G2-S quadrupole time of flight (Q-TOF) mass spectrometer (Waters Ltd.). A reversed-phase column ACQUITY BEH C8 column (1.7 μm, 100 mm×2.1 mm) was used at an operating temperature of 60° C. The aqueous part of the mobile phase consisted of 1 mM ammonia acetate in ultrapure water, pH 4.15. The organic mobile phase was 1:1 isopropanol acetonitrile. For detailed description of the experimental methods see (Sarafian et al., Anal Chem 2015, 87:1171 9662-9670). Data files were imported into MassLynx (Waters Ltd.) where peaks were automatically integrated. Manual inspection on each processed sample file was carried out to ensure that the spectra had been correctly integrated. The extracted peak integral was then normalized to the total ion current (TIC) of each sample.

Metabolomics—SCFA quantification with GC-MS/MS: Colonic biopsies were stored at −40° C. until extraction. Biopsy tissues were transferred to a screw-cap tube and weighed, after which 5 1.0 mm Zirconia beads and 100 μL of ultrapure water were added. The tissue was homogenized 896 in a Biospec bead beater using two 30 seconds cycles at max speed. An eleven-point calibration curve and a pooled QC sample was constructed using genuine SCFA standards. Metabolites were then extracted using methyl tert-butyl ether (MTBE) (Millipore-Sigma) and derivatized with N-tert-butyldimethylsilyl-N-methyltrifluoroacetamide with 1% tert-butyldimethylchloro-silane (MTBSTF+1% TBDMSCI). Samples were analyzed on a 7000D Triple-Quadrupole Gas chromatography-mass spectrometer (GC-MS) (Agilent Technologies Ltd.). Data files were imported and analyzed in MassHunter Workstation Software Quantitation Analysis for QQQ version B.07.01 (Agilent Technologies Ltd.). The resulting SCFA concentration were corrected for dilution factor and normalized by sample weight in Microsoft excel.

Metabolomics—Tryptophan quantification with LC-MS/MS: Stool was weighed on an analytical balance (sample weights ˜50 mg) after which 1 mL of ice cold (−20° C.) extraction solvent with internal standards was added to each sample, and sample mixed by vortexing at max speed for 3-5 seconds. Extraction solvent contained 200 ng/mL tryptamine-d4, 500 ng/mL L-tryptophan-d3, 1000 ng/mL 3-methylindole-d3, 200 ng/mL indole-3-Acetic Acid-d5, 200 ng/mL serotonin-d4 in 80% methanol. Samples were sonicated in a sonication bath at RT for 10 minutes and vortexed. Samples were placed at −80° C. for 1 hour to facilitate protein precipitation. Extracts were cleared of debris via centrifugation at 18,000×g, for 20 minutes at 4° C., and the resulting supernatant was transferred to a new microfuge tube. A quality control sample was prepared by pooling 10 μL of every sample. 100 μL of the sample was transferred to a glass autosampler vial and remaining extracts were stored at −80° C. Standard curves were prepared in 80% methanol in a dilution series from 1000 ng/mL to 0.1 ng/mL.

LC-MS/MS was performed on a Waters Acquity UPLC with T3 C18 stationary phase (1×50 mm, 1.7 μM) column coupled to a Waters Xevo TQ-S triple quadrupole mass spectrometer. Mobile phases were 100% methanol (B) and water with 0.1% formic acid (A). The analytical gradient was: 0 min, 5% B; 0.5 min, 5% B; 2.5 min, 95% B; 3.5 min, 95% B; 3.55 min, 5% B; 5 min, 5% B. Flow rate was 350 μL/min with an injection volume of 2.5 μL. Samples were held at 4° C. in the autosampler, and the column was operated at 45° C. The MS was operated in selected reaction monitoring (SRM) mode. Product ions, collision energies, and cone voltages were optimized for each analyte by direct injection of individual synthetic standards. Inter-channel delay was set to 3 milliseconds. The MS was operated in positive ionization mode with capillary voltage set to 3.2 kV. Source temperature was 150° C. and desolvation temperature at 550° C. Desolvation gas flow was 1000 L/h, cone gas flow was 150 L/h, and argon collision gas flow was 0.2 mL/min. Nebulizer pressure (nitrogen) was set to 7 Bar.

Raw data files were imported into Skyline software (MacLean et al., Ann Med Surg (Lond) 2010, 4:248-253). Each target analyte was manually inspected for retention time and peak area integration. Peak areas were extracted for target compounds detected in biological samples and normalized to the peak area of the appropriate internal standard or surrogate in each sample. Normalized peak areas were exported to Microsoft excel where concentrations were obtained using the linear regression formulas generated from the calibration curve. Limits of detection (LOD) and limits of quantification (LOQ) were calculated as 3× or 10× the standard deviation of the blank divided by the slope of the calibration curve respectively (Broccardo et al., J Chromatogr B Analyt Technol Biomed Life Sci 2013, 934:16-21; Shrivastava and Gupta, Chronicles of Young Scientists 2011, 2:21-25). One compound, 3-methyl-indole, did not produce a linear response and only raw peak areas are reported for this compound (relative quantification only).

Metabolomics data analysis: Metabolomics data was log10 transformed (with pseudo count 1 for zero values) and tested for significance in R with linear mixed-effect models correcting for subject using the lmer function from the lmerTest package with formula lmer(data˜cohort+(1|subject_id). Comparison contrasts were extracted using the get_contrasts function from the psycho package, and nominal p-values were adjusted per contrast table using the p.adjust(, method=“fdr”) function. Significance of by subject-averaged data was tested using Mann-Whitney tests using the wilcox.test function. Time-series analysis was carried out on NMR data using the santaR version 1.0 package in R. The analysis was based on 1,000 bootstrap rounds (95% confidence interval), 1,000 permutation rounds and 4 degrees of freedom.

Cytokine measurements: Multiplexed Luminex according to the manufacturer's instructions was used for quantification of IL-8, IFNγ, IL-10, IL-18, IL-22, Leptin, VEGF, MIG, IL-1β, IL-17A, IL-1RA, IL-6, and TNFα. The beads were recorded on a Bioplex 200 Luminex instrument. Samples were tested in duplicate and values were quantified by interpolation from a 5 point standard curve. TGFβ-1 was quantified using enzyme-linked immunosorbent assay (ELISA) according to the manufacturer's instructions (R&D Systems; Minneapolis, Minn.). Absorbance was measured on a Bio-Rad microtiter plate reader. Samples were assayed in duplicate and values were interpolated from log-log fitted standard curves.

RNA sequencing and analysis: mRNA was extracted from biopsy samples and used for RNA-Seq library preparation following instructions in the Illumina TruSeq RNA Library Prep Kit v2. Sequencing was run on an Illumina High Seq-2000 in the Mayo Clinic Sequencing Core with 101 bp paired end reads. Gene expression counts were obtained using the MAP RSeq v.2.0.0 workflow (Kalari et al., BMC Bioinformatics 2014, 15:224). MAP-RSeq consists of alignment with TopHat 2.0.12 (Kim et al., Genome Biol 2013, 14:R36) against the human hg19 genome build and gene counts with the Subread package 1.4.4 (Liao et al., Nucleic Acids Res 2019, 47:1133 e47). Gene annotation files were obtained from Ensemble version 75. Gene counts were normalized using RPKM (Reads Per Kilobase per million Mapped reads). Differential expression analysis was performed using edgeR 2.6.2 (Robinson et al., Bioinformatics 2010, 26:139-140). Pathway enrichment analyses were performed using R package RITAN (Rapid Integration of Term Annotation and Network resources, bioconductor.org/packages/release/bioc/html/RITAN.html).

Methylome sequencing 988 and analysis: Illumina Infinium MethylationEPIC BeadChips with ˜850K CpG sites were used to assess genome wide methylation in genomic DNA isolated from biopsy samples. For data pre-processing the raw data (.idat) files were loaded into R package ChAMP version 2.9.10 (Tian et al., Bioinformatics 2017, 33:3982-3984). Probes that had detection p-value>0.01, bead count<3, overlapped with SNP sites, or with multiple alignments in the human genome were removed, which resulted in 773,789 CpG sites for downstream analyses. Potential batch effects were corrected by the Combat method (Johnson et al., Biostatistics 2007, 8:118-127; and Sun et al., BMC Med Genomics 2011, 4:84). Differentially methylated CpG sites were detected using the Limma function with Benjamini-Hochberg (BH) multiple testing correction. CpG sites with between-group differential p-value<0.01 and methylation difference greater than 5% were considered as differentially methylated CpGs (DMCs). Clusters within these DMCs (differentially methylated regions (DMRs)) were identified using the Bumphunter algorithm (Jaffe et al., Int J Epidemiol 2012, 41:200-209) and defined as a minimum of 4 probes in the region with adjusted DMR p value<0.05 through permutation test. Genes associated with DMCs or DMRs were used for pathway enrichment analysis with the R package RITAN.

To correct for differences in gender and small numbers of male samples only biopsy samples from female subjects were included for interpretation. Analysis was focused on samples from the first biopsy time point and used time point 2 samples as a verification cohort.

Multi-omics data integration: Association between stool microbial features and stool metabolites was investigated using the Maaslin2 package in R (huttenhower.sph.harvard.edu/maaslin2). Maaslin2 was run using minimum abundance and minimum prevalence for microbial features were set at 0.0001 and 0.5, respectively. Threshold for FDR corrected q-value was set at 0.25. Linear mixed effects models were applied to the association with subject set as random-effect.

Identification of structural deletion and variable regions and subsequent association with significant metabolite features was performed as reported in the 1020 methods paper (Zeevi et al., Nature 2019, 568(7750):43-48). Due to low number of male samples, only data from females was included.

For correlation, networks species were removed that had a mean per-sample abundance under 0.001% after which data was adjusted for compositionality with CLR transformation from the robCompositions package in R. Each set of significant Spearman correlations were assembled into a network using the igraph and plotted using ggraph in R.

Lasso penalized regression machine learning was performed using a model for regularization and feature selection to integrate host gene expression with microbiome and metabolomics data. Host biopsy gene expression from time point 1, collapsed fecal microbiome abundance and collapsed fecal metabolite data were subject-matched, resulting in a subset of 25 IBS patients and 13 healthy controls. The biomaRt R package was used to remove non-protein-coding genes, lowly expressed genes (expressed in less than half of the samples), and genes with low variance, resulting in 12,132 unique genes. A variance stabilizing transformation was performed on the filtered gene expression data using the DESeq2 R package. For the microbiome data, the counts taxa matrix was summarized at species, genus, family, and phylum taxonomic levels, and only taxa found at 0.01% relative abundance in at least 20% of the samples were kept. This filtered taxon matrix was centered log ratio (CLR) transformed. The fecal metabolomic data, NMR metabolites, bile acids, and tryptophan panel data was concatenated, and log2 transformed.

The Lasso regression model was fit separately in order to identify gene and gene-metabolite associations. The gene-wise model uses gene expression for each gene as response and microbiome abundance or metabolite concentrations as predictors. The effect of gender and IBS-subtype was controlled for by including them as binary covariates in our predictor matrix. Leave-one-out cross validation was used for tuning the penalty parameters in the Lasso model fits using the R package glmnet. Inference for Lasso models was performed using regularized projections to obtain significance and confidence interval for each variable associated with a given gene. Multiple hypothesis testing was corrected for using the Benjamini-Hochberg method. Since the Lasso model is sensitive to small variations of the predictor, stability selection was used to select robust variables associated with the host genes. Intersects of outputs from Lasso and stability selection models were inspected and filtered at FDR<0.1. Host gene-gender and host gene-IBS subtype associations were removed.

### Example 2

### Experimental Results

An observational study using longitudinal multi-omics sampling of microbiome and host samples was conducted to identify microbial mediators driving subtype-specific phenotypes in IBS. This design allowed comparison of healthy controls (HC) to patients with IBS-C or IBS-D (Rome III criteria). Study participants were matched for gender, age, and body mass index (BMI). A total of 77 participants provided samples for at least one time point. Of these, a subset of participants agreed to undergo flexible sigmoidoscopy allowing us to obtain colonic biopsies longitudinally. The number and type of samples and distribution of study subjects are outlined in FIGS. 1A and 1B. Study subjects provided dietary recall and symptom severity at each visit. In addition, IBS patients were given the option to provide an additional stool sample at the time of a self-identified flare in between visits, which was characterized as significant worsening of their symptoms. A total of 12 subjects provided the additional stool sample (6 each for IBS-C and IBS-D). As this was optional, it does not imply other subjects did not have a flare in symptoms.

Demographics of the study participants are outlined in TABLE 1. Other information collected included medication use, hospital anxiety, depression score, IBS symptom severity score (SSS), and dietary history, including food frequency questionnaires at the beginning and end of the study, as well as 24-hour dietary recall prior to each fecal sample.

TABLE 1
Cohort description

| | Healthy controls | IBS-C | IBS-D |
|---|---|---|---|
| n | 24 | 23 | 30 |
| mean age (±sd) | 34.5 (±11.0) | 41.6 (±13.6) | 37.4 (±12.3) |
| mean BMI (±sd) | 26.8 (±6.1) | 27.7 (±8.2) | 28.2 (±6.8) |
| female n | 19 | 22 | 20 |
| percentage biopsy | 79.2 | 95.7 | 66.7 |
| biopsy n | 15 | 14 | 13 |
| percentage biopsy | 65.2 | 46.7 | 54.2 |

To identify microbial drivers of subtype-specific symptoms in IBS, shotgun metagenomic sequencing and metabolomics were performed on stool samples, metabolomics and cytokine measurements were performed for serum samples, and 16S rRNA gene-sequencing-based microbial composition, metabolome, transcriptome and methylome analyses were performed for biopsy samples with the details outlined in FIG. 1A.

### Longitudinal Sampling Overcomes Heterogeneity Seen in Cross-Sectional Microbiome Studies

A cross-sectional study of the gut microbiome in chronic GI conditions provides a snap shot of a highly dynamic ecosystem. In addition to the effect of diet, medication use, lifestyle and other environmental factors, the variability in microbiome seen over time also reflects changes in disease activity. The vast majority of microbiome studies in IBS have been cross-sectional, which show limited overlap in terms of compositional changes. The effect of longitudinal sampling on the identification of compositional changes compared to cross-sectional sampling was assessed by subsampling the longitudinal data, testing for significant taxa, and comparing the results with results obtained on data that was averaged across all time points for each subject.

Differences in taxa abundance between HC and disease groups observed in individual time-points were highly inconsistent when comparing the different time points and did not overlap with changes observed in the averaged data (FIG. 2A). When using averaged but not subsampled data, a significantly higher abundance of multiple Streptococcus spp. was found individually in IBS-C and IBS-D, as well as in the composite IBS group, compared to HC (log2(FC)˜1, at FDR<0.25). In addition, a significantly lower abundance of the recently identified phylum Synergistetes was found in IBS-D compared to HC (log2(FC)−2.1, Mann-Whitney FDR 0.017; FIG. 2B). These findings highlighted the importance of longitudinal sampling in chronic diseases to reliably identify microbiota changes that may be misrepresented by cross-sectional sampling. This is further supported by a recent study that shows commonly used ‘omics’ methods are more reliable when using averages over multiple sampling time points (Nature Medicine 2019, 25:1442-1452). Hence, the data presented herein are primarily reported from averaged data per subject.

Principle coordinate analysis (PCoA) based on Bray Curtis β-diversity showed that stool microbiota composition in IBS was clustered by subtype, and IBS-D and IBS-C displayed significantly different dispersion from HC samples as well as from each other (FIGS. 1C and 1D). To further confirm differences in β-diversity, Bray-Curtis dissimilarity (BCD)-based irregularity (BCDI) scores were calculated. BCDI scores for IBS-C were significantly elevated (linear mixed-effect model correcting for subject, IBS-C vs. HC p-value 0.011; FIG. 1E). A sample was considered to be irregular when a disease sample was beyond the 90^{th} percentile of the HC distribution. It was observed that more IBS-C samples were irregular than IBS-D (31.7% for IBS-C, 14.1% for IBS-D).

### Longitudinal Sampling Revealed Greater Variability in IBS-C Microbiota Over Time

The stool microbiota composition exhibited significantly greater variability over time in patients with IBS-C compared to IBS-D (FIG. 1F) (mean within subject Bray Curtis distance, within-D vs. within-C, Tukey ANOVA q-value<0.005). In addition, there was higher α-diversity in averaged IBS-C stool samples compared to IBS-D samples (ANOVA with Tukey HSD p-value 0.016).

Differences in luminal and mucosa-associated microbiota are relevant in IBS as disease subtypes, defined by differences in stool form, are partly the result of alteration(s) in epithelial function. The microbial composition in the colonic mucosa typically is significantly different from the luminal microbiota in stool samples (Bray Curtis β-diversity, biopsy vs. luminal PERMANOVA p-value 0.001; FIG. 1G). The mucosa-associated microbiota in IBS patients was characterized by significantly higher levels of Proteobacteria compared to HC (log2(FC) 0.4, Mann-Whitney FDR 0.23; FIG. 2C), and this was true across both time points. The mucosa-associated microbiota in patients with IBS-C was more dissimilar from its respective luminal microbiota than those of IBS-D or HC (FIG. 1H). In addition, there was greater intra-individual variability in mucosa-associated microbiota in patients with IBS-C across time similar to what was observed in the luminal microbiota (FIG. 1I).

### IBS Symptom Severity was Associated with Functional Changes in the Gut Microbiota

The severity of IBS at particular sampling points was reported using the IBS symptom severity score (SSS, range 0-500), which is a cumulative metric of abdominal pain intensity, frequency, distension, dissatisfaction with bowel habits, and influence of IBS on life in general. A higher relative abundance of more than 20 Lactobacillus spp. was observed in severe IBS-D (SSS>300) compared to mild-moderate IBS-D (SSS<300; >10-fold, Mann-Whitney FDR<0.1). Interestingly, this was not related to probiotic consumption by the subjects. When considering functional variation through Kyoto Encyclopedia of Genes and Genomes (KEGG) ontology (KO) term abundance in the stool metagenomics data, it was found that 74 KO terms were associated with severe IBS-C, and 44 with severe IBS-D at an FDR of <0.1. The KO term for alcohol dehydrogenase was found in both severe IBS-C and IBS-D compared to mild-moderate IBS (˜0.6 log2(FC) higher in severe IBS), suggesting a potential relationship to abdominal pain that is common to both IBS-C and IBS-D.

### Integrated Top-Down Bottom-Up Approach Provides Mechanistic Insight Into the Effect of Gut Microbiota Metabolism on Host Physiology

To better understand the mechanism by which the gut microbiota can drive symptom pathophysiology, the metabolic output of the microbiome reflected in the biochemical profiles of the luminal and mucosa-associated samples was investigated.

Studies first focused on microbiota-derived metabolites that can drive changes in gastrointestinal physiology relevant to IBS. ¹H-nuclear magnetic resonance (NMR) spectroscopy identified the SCFA propionate, butyrate, and acetate as being significantly lower in the stool samples of patients with IBS-C compared to HC (log2(FC) −0.38, −0.54, −0.56 respectively, linear mixed-effect model correcting for subject, IBS-C vs. HC p-value<0.01); see, FIGS. 3A and 4A for averaged data. Consistent with the luminal metabolites, acetate (measured by gas chromatography-mass spectrometry (GC-MS)) was also significantly reduced in the colonic mucosal biopsy samples from the IBS-C group compared to the HC group (FIG. 3B). Notably, these differences in SCFA were independent of the overall intake of dietary fiber, as this was not significantly different between the groups.

The role of SCFA in modulating the serotonergic pathway in host GI tissue has been described (Bhattarai et al., Am J Physiol Gastrointest Liver Physiol 2017, 313(1):G80-G87; and Reigstad et al., FASEB J2015, 29(4):1395-1403). Thus, to determine the physiologic relevance of lower SCFA in stool and biopsies seen in IBS-C patients, the change in short circuit current (ΔIsc; a measure of ionic flux across the epithelium reflecting intestinal secretion) was investigated in colonic epithelium in response to serotonin (5-HT) using an Ussing chamber setup. Consistent with the observed decrease in SCFA in IBS-C, the secretory response of colonic biopsies from IBS-C patients was significantly lower than HC (FIG. 3C). This highlighted the importance of concurrent top-down bottom-up approaches to determine mechanisms identified in animal studies that are relevant in human disease states.

The bacterially-derived monoamine, tryptamine (a tryptophan metabolite similar to serotonin), can activate serotonin receptor-4 (5-HT₄R), resulting in increased colonic secretion in gnotobiotic mice (Bhattarai et al., Cell Host Microbe 2018, 23(6):775-785.e5), but its physiologic role in human disease has not yet been determined. Thus, studies were conducted to investigate changes in tryptamine and other tryptophan metabolites in stool samples using a targeted LC-MS method. It was found that both tryptophan and tryptamine were significantly increased in stool samples from IBS-D patients (FIGS. 3E and 4B). Again, this was not related to dietary differences in protein intake. To determine the physiologic relevance of differences in tryptamine level, differences in Isc were measured in colonic biopsies obtained from the three groups. Consistent with the findings in gnotobiotic mice, colonic biopsies from IBS-D patients (FIG. 3D) also exhibited significantly higher baseline Isc. This also was consistent with the secretory effect of tryptamine.

In humans, the primary bile acids (BA) cholic acid (CA) and chenodeoxycholic acid (CDCA) are deconjugated from their glycine or taurine conjugates by microbial bile salt hydrolase (BSH) activity. These primary BAs then serve as substrates for a diverse range of microbial modifications, including conversion to the secondary bile acids deoxycholic acid (DCA) and lithocholic acid (LCA), and de-sulfation of DCA-S to DCA. Certain forms of bile acids (e.g., hydroxylated bile acids) can increase colonic secretion in humans. Studies therefore were conducted to determine whether there are differences in microbial biotransformation of bile acids in IBS that may contribute to altered intestinal secretion. Variation in BA signatures associated with IBS were identified (liquid chromatography-mass spectrometry (LC-MS/MS)), with significantly higher levels of the unconjugated primary BAs in stool samples from patients with IBS-D and significantly lower levels of unconjugated primary BAs in stool samples from IBS-C (FIGS. 3F, 4C, and 4D) compared to HC. Higher levels of individual primary bile acids and DCA-S also were observed in IBS-D compared to HC and IBS-C subjects (FIGS. 4C and 4D). Since hydroxylated primary bile acids such as CDCA may increase colonic secretion, the effect of CDCA was tested in colon mucosa-submucosa preparations from germ-free mice in an Ussing chamber set-up. Indeed, a significant increase in Isc in response to CDCA was observed (FIG. 4E), which further supported a physiological role for the increased CDCA in IBS-D patients.

Current paradigms implicate bile acid malabsorption as a driver of increased secretion in IBS-D patients but this should result in an increase in both primary and secondary bile acids. Notably, no significant differences were observed in DCA or LCA among the groups, suggesting that bile acid malabsorption alone cannot account for the effects of bile acids and decreased microbial biotransformation is at least partly responsible for the bile acid effects seen in IBS-D patients.

### Integrated Microbiome-Metabolome Analysis Identifies a Novel Microbial Metabolic Pathway in IBS

In addition to the above targeted approach, an untargeted metabolomics approach was employed to identify novel microbial pathways that may be driving pathophysiologic changes in IBS. A projection to latent structures-discriminant analysis (PLS-DA) model based on untargeted ¹H-NMR spectral profiles identified metabolic variation between the IBS subgroups and HC samples (FIGS. 5A-5C and 6A-6C). Lysine, uracil and hypoxanthine were all found to be significantly lower in stool samples from IBS-C patients compared to HC. Hypoxanthine was also lower in IBS-D patients, although not at the same significance as in IBS-C. Hypoxanthine can serve as an energy source for intestinal epithelial cells and promotes intestinal cellular barrier development and recovery following injury or hypoxia (Lee et al., J Biol Chem 2018, 293(16):6039-6051). Lower hypoxanthine levels in the stool can reflect decreased production or elevated breakdown by the microbiome in the gut of IBS patients.

To gain insights into possible microbial contributions to the fecal hypoxanthine pool, metagenomics modules related to hypoxanthine were interrogated in stool samples from patients with IBS and HC. Among the KO terms, it was found that xanthine dehydrogenase/oxidase (XO) and xanthine phosphoribosyltransferase (XPRT) were elevated in IBS-C and IBS-D compared to HC [XO: 1.17.1.4 (multiple KO terms annotated); FIG. 5D, log2(FC) 0.73, p-value<0.005, q-value 0.09 for IBS-C and log2(FC) 0.49, p-value<0.07 for IBS-D. XPRT: 2.4.2.22, log2(FC) 1.30, p-value<0.05, q-value 0.12 for IBS-C, log2(FC) 0.64, p-value<0.07 for IBS-D]. XPRT liberates xanthine from xanthosine-monophosphate as an early step in purine salvage. Downstream, XO is an enzyme with low substrate specificity that acts on xanthine or hypoxanthine to produce uric acid. Higher levels of these XPRT and XO modules suggest increased purine breakdown by gut microbiota in IBS patients.

The metagenomic KO terms were inspected further to explore two aspects of hypoxanthine metabolism—namely its role in modulating the epithelial energy state, and generation of H₂O₂ and superoxide anions given the putative role in IBS (Med Sci Monit 2013, 19:762-766). Related to energy metabolism, four modules from the tricarboxylic acid cycle (TCA) cycle (L-lactate-dehydrogenase, pyruvate-dehydrogenase, formate dehydrogenase, and fumarate hydratase) and four terms for alternative forms of respiration (sulfite reductase/ferredoxin, sulfite-, nitrite reductase, and cytochrome-C oxidase) were significantly elevated in IBS-C stools compared to HC (<q-value 0.1). Interestingly, the superoxide reductase (1.15.1.2) term was elevated in IBS-C (log2(FC) 0.54, p-value<0.005, q-value 0.09), which could reflect increased capacity to deal with oxidative stress in the IBS-C gut microbiome. This might be necessary in situations of high XO activity. Together, these data suggested that the microbiome in IBS patients exhibits an increased capacity for hypoxanthine utilization and breakdown, which is congruent with the lower hypoxanthine levels in IBS-C stools. As hypoxanthine promotes the intestinal barrier, differences in paracellular flux across the colonic epithelium were examined using colonic biopsies, demonstrating higher intestinal permeability in biopsies obtained from IBS-C patients compared to HC.

### Microbial Gene Regions Contribute to Variation of Microbial Metabolites in IBS

To further elucidate the microbial contribution to differential metabolite abundances identified in IBS, direct multivariate correlation analysis based on linear models was first performed (Maaslin; huttenhower.sph.harvard.edu/maaslin2). This identified 60 significant metabolite-species correlations for HC samples, 28 for IBS-C, and 46 for IBS-D (FIGS. 7A-7C). No correlations were present in all categories, but 12 were present in HC, IBS-C, or IBS-D. Two correlations were present in both the IBS-C and IBS-D subgroups (FIGS. 7D and 7E).

While the above correlational approach allowed identification of potential microbial drivers of differences in fecal metabolites, it was not able to identify specific microbial genes that might be relevant for the differences in detected metabolites. Studies thus were conducted to look for specific bacterial genomic regions that may be responsible for the variation in metabolic output between the groups using a method that associates structurally variable genomic regions to metabolite abundances (SV association; Zeevi et al., supra). This analysis allowed identification of microbial genes involved in the production or consumption of metabolites by either identifying deletion regions (DRs) that are completely missing from some microbiomes or variable regions (VRs) that display variable abundance in some microbiomes.

Sixteen (16) DRs and 20 VRs that correlate with, respectively, 9 and 8 metabolites at a q-value<0.1 were identified. All DRs are from a single bacterium, Blautia wexlerae DSM19850, and exclusively contain genes of unknown function. CDCA is the most frequently associated metabolite, as it covaries with 4 DRs and 7 VRs. This is followed by CA, with 3 DRs and 7 VRs. The multitude of associations with bile acids could reflect presently unknown genes that are involved in modification of primary bile acids.

The strongest observed correlation for the VRs is for a single region from Lachnospiraceae bacterium 3 1 46FAA with hypoxanthine (R 0.70, q-value 0.02) (FIG. 8A). pBLAST analysis perfectly matches the CDS with topoisomerase III (E value=0). As hypoxanthine is a precursor for energy metabolites and topoisomerase III is linked to DNA replication, this may indicate increased utilization of hypoxanthine for growth.

Two regions from Blautia obeum ATCC 29174 were present at significantly lower levels in IBS-C samples, and the regions were positively correlated with butyrate (FIG. 8B). This is consistent with Blautia spp. being a butyrate producer and the lower butyrate levels in IBS-C. These regions are 2676-2677, which encodes a tetricoat peptide, and 2704-2705, which is annotated as a type III ribonuclease. The above examples underscore the increased resolution that can be achieved using SV association analysis and will allow additional work to focus on specific microbial gene regions as potential targets.

### Alteration in Gut Microbiome and Microbial Metabolites Underlie Flares in IBS Patients

IBS is a chronic disease with temporal variability in symptom severity, where most patients will experience transient worsening of symptoms. The longitudinal analysis described herein using the variability in symptoms and microbiome among the groups identified a potential link between the gut microbiome and symptom severity in IBS patients. To further determine if there is a microbial basis for potential exacerbation in symptoms, studies were conducted to look specifically at additional samples collected at the time of self-reported worsening of symptoms (flare) in the subset of patients who provided an additional sample. The flare samples exhibited a higher Bray-Curtis dissimilarity-based irregularity (BCDI) compared to the baseline (averaged non-flare) IBS samples (linear mixed-effect model correcting for subject, p-value 0.011; FIG. 9A). Comparison of within-disease flare samples showed both significantly higher BCDI (FIG. 9B), and lower Shannon α-diversity in flare samples when compared to baseline (averaged non-flare) samples from the respective IBS subgroup (FIG. 9C). Specific bacterial taxa are significantly associated with flares, both when considering IBS patients as one group, as well as within IBS-D and IBS-C patients (168 species for IBS together, 40 for IBS-C, and 7 species for IBS-D at q-value<0.1 from Mann-Whitney test compared to all respective baseline samples). These species almost exclusively decrease in abundance during flare episodes. However, one species of Archaea, Halobiforma nitratireducens, was consistently elevated in the flare samples (FIG. 9D). This Archaeon is capable of nitrate reduction, an alternative form of respiration that was identified above to be among the energy metabolism-related KO terms that are present at higher abundance in IBS-C.

Primary bile acids were significantly higher only in IBS-D flares (linear mixed-effect model compared to baseline and correcting for subject, q-value<0.005) and given the secretory role of CDCA, this may contribute to increased colonic secretion during symptom exacerbation (FIGS. 9E, 9F, and 3E). Functional metagenomic KO modules associated with flares also were investigated, with a focus on modules that had been identified as being associated with symptom severity, as well as the newly implicated hypoxanthine metabolic pathway. Of these, alcohol dehydrogenase and XO were found at higher abundance in IBS-D flares (log2(FC) 0.78, q-value 0.147, p-value<0.02; XO; log2(FC) 1.36, q-value 0.147, p-value<0.02), which again coincided with increases in the TCA cycle and respiration terms identified above. This is further consistent with the findings described above linking IBS symptom severity with the gut microbiome.

To identify possible microbial trajectories that lead to a flare, microbiome changes in individual patients were analyzed. An IBS-C patient was identified in whom time-course permutation analysis revealed a significant increase in BCDI over time, culminating in a flare episode (FIG. 10A). For the 10 remaining flare patients with more than two time points, four flare samples also showed the greatest BCDI compared to their baseline. In one IBS-D patient, this was explained by an increase predominantly in Streptococcus spp. (FIGS. 10B and 10C), and at the functional level this flare sample displayed strongly elevated levels of the secretory metabolites tryptamine, CA, and CDCA, as well as a reduction in the bile salt hydrolase KO term (elevated and decreased defined as |Z-score|>1.645 corresponding to an α of <0.05) (FIG. 10D). These observations highlighted the potential for unique microbial and metabolic features to potentially contribute to worsening symptoms.

### Epigenetic and Transcriptomic Differences Reflecting Altered Host Gastrointestinal Function in IBS

As for most chronic conditions, the pathophysiology of IBS is multifactorial, with contributions from host and microbial pathways as well as host-microbial co-metabolism. To determine the effect of microbial metabolism on host function, transcriptional and epigenetic changes in colonic biopsies were evaluated. To correct for differences in gender and small numbers of male samples, only biopsy samples from female subjects were included for interpretation. The analysis was focused on samples from the first biopsy time point, and time point 2 samples were used as a verification cohort.

Eight-two (82) differentially expressed (DE) genes were identified when comparing IBS-C and HC (FIGS. 11A and 11C), and 78 DE genes were identified when comparing IBS-D and HC (FIGS. 11B and 11C) (>1 absolute log2(FC) change and p-value<0.05). The overlap of these two sets of significant DE genes was 20.8% for the IBS-C comparison and 21.8% for the IBS-D comparison. Using KEGG pathway enrichment analysis, it was observed that immune and inflammation-related pathways were enriched in IBS patients, such as the B cell receptor signaling pathway, primary immune deficiency pathway, antigen processing and presentation and complement and coagulation cascades pathway (FIG. 11E). Since transcriptome differences were observed in immune pathways, cytokines were assessed both in serum and colonic biopsies to determine if there were IBS-specific immune changes. A significant difference was not found for any of the pro-inflammatory cytokines in either blood or colonic biopsies in patients with IBS.

An epigenome analysis focused on differentially methylated regions (DMRs). Fifty-four (54) DMRs were detected when comparing IBS-C and HC, and 75 DMRs when comparing IBS-D vs. HC (FIG. 11D) (DMR p-value<=0.02 from permutation test). KEGG pathway enrichment analysis on the DMR-associated genes showed that the antigen processing and presentation pathway was enriched in both comparisons, but the enrichment was driven by different genes (HLA-B and HLA-DQB1 for IBS-C, and HSPA1A, HLA-F and HLA-B for IBS-D) in the IBS subtypes.

To determine if some of the transcriptional differences were driven by differentially methylated regions, the overlap between the two was examined, resulting in identification of antigen processing and presentation as one such pathway. It was interesting to note that while HLA genotype and HLA-DQ genes have been previously associated with celiac disease and inflammatory bowel disease, they have not been described in IBS (Ann Med Surg (Lond) 2015, 4(3):248-253; and World J Gastroenterol 2018, 24(1):96-103), highlighting a potential predisposing locus in IBS.

### Targeted Multi-Omics Integration Identifies Purine Starvation in Colonic Epithelium as a Potential Novel Mechanism Underlying IBS

The -omics data were integrated to better understand the observed changes in biology. In examining the microbiome and the metabolome, it was observed that fecal hypoxanthine abundance was significantly lower in IBS-C and IBS-D, as described above. At the functional level, the changes were consistent with increased purine degradation by the microbiome. However, hypoxanthine is a host-microbial co-metabolite, and while fecal abundance is predominantly influenced by the microbiome, it can also be affected by host metabolism. To determine the host contribution to the hypoxanthine pool, changes in purine metabolism gene expression in colonic biopsies were examined. The human xanthine oxidase (XDH gene) was elevated in IBS subtypes when compared to HC, and this was seen at both time points with a log2 (FC) ranging between 0.26 and 0.65 (FIG. 12C; nominal p-values 0.02 for IBS-C and 0.10 for IBS-D in time point 1, and <0.005 for the comparisons at time point 2). This suggested that depletion of the hypoxanthine pools may be a result of increased XO activity from both the microbiome and the host.

Intestinal epithelial cells have limited capacity for de novo synthesis of purines, and instead rely on salvage pathways for adenylate biosynthesis. Thus, to identify secondary effects on the host resulting from depletion of hypoxanthine pool, transcriptional changes in the purine salvage pathway were examined. Purine nucleoside phosphorylase (PNP), the first gene in the purine salvage pathway, was expressed at an about 2-fold higher level in both IBS-C and IBS-D (log2(FC) 0.50 and 0.65, q-value 0.118 and 0.018, p-value<0.001 for IBS-C and IBS-D respectively), and even within IBS patients, PNP expression was lower in samples with comparatively higher hypoxanthine levels (FIG. 12C).

Together, these findings suggested a model in which elevated degradation of purine nucleotides by the microbiota and the host induces metabolic stress in colonic tissue. In turn, this leads to a compensatory response by increasing purine salvage. Low levels of purine nucleotides result in lower epithelial energy state and capacity for mucosal repair, which may underlie the pathophysiology of IBS.

### Integrated Multi-Omics Analysis Points to Microbiome-Host Interactions in IBS

Further studies were conducted to search for putative host-microbial-metabolite interactions in an untargeted way by constructing a cross-omics data type association network. Two correlation networks were built—the first including biopsy microbiome, metabolomics, and transcriptomics data (biopsy network; FIG. 13A), and the second with luminal microbiome and metabolomics data with host transcriptome (luminal network; FIGS. 13B and 13C). However, such correlation networks are difficult to interpret in the context of biological differences, and the edges in both of these networks were present only at high false discovery rates (<0.25 FDR).

A more powerful machine learning-based integration approach was therefore employed by applying a Lasso penalized regression model to obtain more insight in covarying features across the -omics data sets. This approach was used to look for general gene-transcript and gene-metabolite associations at an FDR<0.1 separately for HC and IBS. In IBS samples, 688 unique host genes associated with 30 fecal metabolites were identified (FIG. 13D), along with 328 unique host genes associated with 99 bacterial taxa (FIG. 13E) at an FDR<0.1. In HC samples, 272 unique host genes associated with 26 fecal metabolites were identified, along with 50 genes associated with 34 microbial taxa. The top 20 canonical enriched gene pathways associated with fecal metabolite levels for IBS samples are shown in FIG. 13F, while the top 20 canonical enriched gene pathways associated to microbial taxa for IBS samples are shown in FIG. 13G. None of the gene-taxa and only 3 gene-metabolite associations were seen in both HC and IBS, illustrating the different metabolic modes of the IBS microbiome.

In addition, 33 genes were identified as being associated with both fecal metabolites and microbial taxa at FDR<0.25 (FIG. 12A). In this network, an interesting hub was seen with acetate and the PGLYRP1 and KIFC3 genes (FIG. 12B). PGLYRP1, a pattern receptor that binds to murein peptidoglycans (PGN) of Gram-positive bacteria and leads to bactericidal activity through interference with peptidoglycan biosynthesis, was also negatively correlated with the broad family of Gram-positive bacteria Peptostreptococcaceae. KIFC3 is a minus-end microtubule-dependent motor protein required for maintenance for zonula adherens with potential impact on intestinal barrier function.

Taken together, the studies described herein provide findings from integrated longitudinal multi-omics analysis of the gut microbiome, metabolome, host epigenome, and transcriptome in the context of host physiology in patients with IBS. Longitudinal sampling was found to overcome the heterogeneity typically seen in cross-sectional microbiome studies. Robust changes in the gut microbiota composition and diversity in IBS subtypes were observed, underscoring the importance of longitudinal sampling in chronic GI diseases with fluctuating symptoms. The symptom severity in IBS patients was associated with changes in the gut microbiome and similar changes seen in patients with self-identified flares, further validating the importance of these changes. Interestingly, individual-specific changes in gut microbiome and metabolome were noted at the time of flares, which is not surprising given the heterogeneous presentation and multiple physiological processes implicated in IBS. An integrated top-down bottom-up approach was used to determine the relevance of previously identified physiological effects of gut microbial metabolites in rodent models, such as the effect of SCFA on host serotonergic pathways, and effect of tryptamine and CDCA on intestinal secretion in the context of subtype specific phenotypes in IBS. Specific microbial gene regions were identified in Blautia obeum and Lachnospiraceae bacterium 3 1 46FAA that are related to changes in butyrate and hypoxanthine respectively, using a method that associates structurally variable genomic regions to metabolite abundances.

Further, using integration of multiple host and microbiome data layers, purine metabolism resulting in decreased hypoxanthine pool was identified as a novel host-microbial metabolic pathway in IBS, with potential effects on gastrointestinal barrier function and oxidative stress. Interestingly, a similar pathway has been implicated in driving severity in inflammatory bowel disease (Sci Transl Med 2017, 9(380): eaaf9044). This is an appealing target given the availability of a xanthine oxidase inhibitor (allopurinol) which is currently used to treat gout and could potentially be used in a subset of patients with IBS.

A combination of targeted and untargeted approaches were used in the longitudinal multi-omics analysis described herein, which allowed discovery of novel pathways. Several novel integration tools were used that significantly enhanced the ability to focus on a more narrow set of pathways having potential biological significance in IBS. As an example, in addition to pair-wise correlations, a powerful machine learning-based integration approach was used by applying a Lasso penalized regression model to identify gene-metabolite-microbiome relationships with potential implications in the pathophysiology of IBS.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method for treating a mammal identified as having irritable bowel syndrome (IBS), wherein the method comprises treating the mammal to increase hypoxanthine levels in the mammal.

2. The method of claim 1, wherein the treating comprises administering to the mammal an agent effective to increase levels of hypoxanthine in the mammal.

3. The method of claim 2, wherein the agent is hypoxanthine.

4. The method of claim 2, wherein the agent is an inhibitor of xanthine oxidase.

5. The method of claim 5, wherein the inhibitor of xanthine oxidase is allopurinol.

6. The method of claim 2, wherein the agent comprises at least one live bacterial organism having the ability to produce hypoxanthine.

7. The method of claim 6, wherein the at least one live bacterial organism is Escherichia coli K12, an Enterococcus sp, a Faecalibacterium sp, a Bacillus sp., or Bacteroides thetaiotaomicron engineered to produce hypoxanthine.

8. The method of claim 1, wherein the treating comprises selectively removing from the mammal at least one bacterial organism having xanthine oxidase activity.

9. The method of claim 8, wherein the at least one bacterial organism is a Lachnospiraceae spp. or Hungatella hathewayi.

10. The method of claim 8, wherein the method comprises selectively removing said at least one bacterial organism by administering a bacteriophage or an antimicrobial compound.

11-13. (canceled)

14. The method of claim 4, further comprising administering to the mammal at least one live bacterial organism having tryptophan decarboxylase activity.

15. The method of claim 14, wherein the at least one bacterial organism is a Prevotella sp., a Bacteroides sp., a Clostridium sp., a Faecalibacterium sp., a Eubacterium sp., a Ruminococcus sp., a Peptococcus sp., a Peptostreptococcus sp., a Bifidobacterium sp., an Escherichia sp., a Lactobacillus sp., an Akkermansia sp., or a Roseburia sp.

16. (canceled)

17. The method of claim 4 or claim 8, further comprising administering to the mammal at least one live bacterial organism having the ability to produce short chain fatty acids.

18. The method of claim 17, wherein the short chain fatty acids comprise acetate and/or butyrate.

19. The method of claim 18, wherein the at least one live bacterial organism is Faecalibacterium prausnitzii (clostridial cluster IV), an Anaerostipes sp., a Eubacterium sp., a Roseburia sp. (clostridial cluster XIVa), a Blautia sp., a Bifidobacteria sp., a Lactobacillus sp., Akkermansia muciniphila, a Prevotella sp., or a Ruminococcus sp.

20. The method of claim 4 or claim 8, further comprising administering to the mammal at least one live bacterial organism having the ability to convert primary bile acids to secondary bile acids.

21. The method of claim 20, wherein the at least one bacterial organism has the ability to convert cholic acid to deoxycholic acid and/or the ability to convert chenodeoxycholic acid to lithocholic acid.

22. The method of claim 21, wherein the at least one bacterial organism is a Clostridium sp.

23-24. (canceled)

25. The method of claim 1, wherein the agent is administered orally or rectally.

26. (canceled)

27. The method of claim 1, wherein the administering is effective to reduce one or more symptoms of IBS in the mammal.

28-29. (canceled)
